(19) United States
(12) Patent Application Publication
(10) Pub. No.: US 2026/0237458 A1
(43) Pub. Date: Aug. 13, 2026

(54) METHODS FOR DETECTING AND CHARACTERIZING MICROSATELLITE INSTABILITY WITH HIGH THROUGHPUT SEQUENCING

(71) Applicant: Sophia Genetics S.A., Rolle (CH)

(72) Inventors: Lin SONG, Renens (CH); Xiaboin XING, Rolle (CH); Zhenyu XU, Nyon (CH); Frédéric Michaud, Lausanne (CH)

(73) Assignee: Sophia Genetics S.A., Rolle (CH)

(21) Appl. No.: 19/629,843

(22) Filed: Mar. 26, 2026

Related U.S. Application Data
(63) Continuation-in-part of application No. 17/438,462, filed on Feb. 5, 2021.

(30) Foreign Application Priority Data
Feb. 7, 2020 (EP) 20156032.3

(51) Int. Cl. G16B 20/20; C12Q 1/6855; C12Q 1/6869; C12Q 1/6886; G16B 30/10

(52) U.S. Cl. CPC G16B 20/20; C12Q 1/6855; C12Q 1/6869; C12Q 1/6886; G16B 30/10; C12Q 2600/156

(57) ABSTRACT

The proposed methods facilitate the deployment of high-throughput genomic data analysis testing for large pools of patients with comparable sensitivity and specificity to other assays. The characterization, classification, and reporting of MSI status of patient genomic samples may be provided by high throughput genomic analysis of a set of microsatellite marker loci. A multi-parametric background model may be used to infer at least two parameters respectively characterizing a sample variant fraction and MSI genomic alterations of a patient DNA sample relative to a reference background model of the MSS homopolymer length distribution, without the need to use a germline control sample. A local MSI score may be calculated as a function of the at least two parameters to characterize the MSI status at each locus, and a global composite MSI score may be calculated over all tested loci to characterize and report the overall MSI status for the patient sample.

### CROSS REFERENCE TO RELATED APPLICATIONS

The present application is a continuation-in-part application which claims the benefit of U.S. patent application Ser. No. 17/438,462 for METHODS FOR DETECTING AND CHARACTERIZING MICROSATELLITE INSTABILITY WITH HIGH THROUGHPUT SEQUENCING, filed on Sep. 12, 2021; International Application No. PCT/EP2021/052880, for METHODS FOR DETECTING AND CHARACTERIZING MICROSATELLITE INSTABILITY WITH HIGH THROUGHPUT SEQUENCING, filed on Feb. 5, 2021; and European Patent Application No. EP20156032.3, for METHODS FOR DETECTING AND CHARACTERIZING MICROSATELLITE INSTABILITY WITH HIGH THROUGHPUT SEQUENCING, filed on Feb. 7, 2020, the entire contents of which are incorporated herein by reference.

### FIELD OF THE INVENTION

Methods described herein relate to genomic analysis in general, and more specifically to next generation sequencing applications.

### BACKGROUND OF THE INVENTION

### Next-Generation Sequencing

High throughput sequencing (HTS), next-generation sequencing (NGS) or massively parallel sequencing (MPS) technologies have significantly decreased the cost of DNA sequencing in the past decade. NGS has broad application in biology and dramatically changed the way of research or diagnosis methodologies. For example, RNA expression profiling or DNA sequencing can only be conducted with a few genes with traditional methods, such as quantitative PCR or Sanger sequencing. Even with microarrays, profiling the gene expression or identifying the mutation at the whole genome level can only be implemented for organisms whose genome size is relatively small. With NGS technology, RNA profiling or whole genome sequencing has become a routine practice now in biological research. On the other hand, due to the high throughput of NGS, multiplexed methods have been developed not just to sequence more regions but also to sequence more samples. Compared to the traditional Sanger sequencing technology, NGS enables the detection of mutations for much more samples in different genes in parallel. Due to its superiorities over traditional sequencing method, NGS sequencers are now replacing Sanger in routine diagnosis. In particular, genomic variations of individuals (germline) or of cancerous tissues (somatic) can now be routinely analyzed for a number of medical applications ranging from genetic disease diagnostic to pharmacogenomics fine-tuning of medication in precision medicine practice. NGS consists in processing multiple fragmented DNA sequence reads, typically short ones (less than 300 nucleotide base pairs). The resulting reads can then be compared to a reference genome by means of a number of bioinformatics methods, to identify specific subsequences carrying small variants such as Single Nucleotide Polymorphisms (SNP) corresponding to a single nucleotide substitution, as well as short insertions and deletions (INDEL) of nucleotides in the DNA sequence compared to its reference.

### NGS Workflow Automation and Optimization

NGS enables in particular the detection and reporting of small changes in the DNA sequence, such as single nucleotide polymorphisms (SNPs), insertions or deletions (INDELs), as compared to the reference genome, through bioinformatics methods such as sequencing read alignment, variant calling, and variant annotation. NGS workflows refer to the configuration and combination of such methods into an end-to-end genomic analysis application. In genomic research practice, NGS workflows are often manually setup and optimized using for instance dedicated scripts on a UNIX operating system, dedicated platforms including a graphical pipeline representation such as the Galaxy project, and/or a combination thereof. As clinical practice develops, NGS workflows may no longer be experimentally setup on a case-per-case basis, but rather integrated in Saas (Software as a Service), PaaS (Platform as a Service) or IaaS (Infrastructure as a Service) offerings by third party providers. In that context, further automation of the NGS workflows is key to facilitate the routine integration of those services into the clinical practice.

While next generation sequencing methods have been shown more efficient than traditional Sanger sequencing in the detection of SNPs and INDELs, their specificity (rate of true positive detection for a given genomic variant) and sensitivity (rate of true negative exclusion for a given genomic variant) may still be further improved in clinical practice. The specificity and sensitivity of NGS genomic analysis may be affected by a number of factors:
(1) Biases introduced by the sequencing technology, for instance due to:
   (i) Length of the reads relative to the length of the fragments;
   (ii) Too low number of reads (read depth);
   (iii) Errors or low quality bases introduced during sequencing; and
   (iv) Inherent difficulties in counting homopolymer stretches, resulting in insertion and deletion errors;
(2) Biases introduced by the DNA enrichment technology, for instance due to:
   (i) Primers or probes non-specific binding, for instance due to storing the assay at a low temperature for too long, or due to too small amount of DNA in the sample;
   (ii) Introduction of sequence errors caused by imperfect PCR amplification and cycling, for instance due to temperature changes;
   (iii) Suboptimal design of the probes or primers. For example, mutations may fall within the regions of the probes or primers;
   (iv) Enrichment method limitations. For instance, long deletion may span the amplified region;
   (v) Cross-contamination of data sets, read loss and decreased read quality due to fragment tagging with barcodes, adapters and various pre-defined sequence tags; and
   (vi) Chimeric reads in long-insert pair-ended reading.
(3) Biases introduced by the sample itself, for instance due to:
   (i) Somatic features, in particular in cancer diagnosis based on tumor sample sequencing; and
   (ii) The type of biological sample, e.g. FFPE, blood, urine, saliva, and associated sample preparation issues, for instance causing degradation of DNA, contamination with alien DNA, or too low DNA input.
(4) Biases introduced by the genomic data structure of certain regions specifically, for instance due to:
   (i) High ratio of GC content in the region of interest;
   (ii) Presence of homopolymers and/or heteropolymers, that is partial genomic sequence repetitions of one or more nucleotides in certain regions, causing ambiguities in initial alignment, and possibly inherent sequencing errors;
   (iii) Presence of homologous and low-complexity regions; and
   (iv) Presence of non-functional pseudogenes that may be confused with functional genes, in particular in high-repeat genomic regions of the human genome when the DNA fragments are not long enough compared to the read length.

This limits the efficient deployment of NGS in routine genomic analysis applications, as a different genomic data analysis workflow needs to be manually organized and configured with different sets of parameters, by highly specialized personnel for each application to meet the clinical expectations in terms of specificity and sensitivity. The automation of genomic data processing workflows is particularly challenging as the workflows need to be adapted to the specific data biases introduced by the upstream NGS biological processes on the one hand and the genomic data structures inherent to the current application on the other hand. In early deployment of genomic testing, a limited number of tests and setups were processed by dedicated platforms, which could be manually setup, configured and maintained by highly skilled specialized staff. This approach is costly and does not scale well as more and more tests have to be conducted in daily operation by a single multi-purpose genomic analysis platform.

### NGS Analysis of Homopolymer and Heteropolymer Variants

In terms of automating the NGS analysis, special attention needs to be devoted to the inherent difficulties in characterizing the indel variants in homopolymer and/or heteropolymer regions of the reference human genome. Mischaracterization of some homopolymer or heteropolymer variants may result in false positive or false negative detection of certain traits and diseases in a diversity of diagnosis applications.

A statistical method to better detect insertions and deletions in a homopolymer region and detecting the corresponding heterozygosity is described in US patent application 2014/0052381 by Utiramerur et al. They observed that in an NGS genomic analyzer workflow, the read alignment is not necessarily correct, but it may be possible to determine the heterozygosity from the distribution of base calling residuals based on measured and model-predicted values in the homopolymer regions by using a Bayesian peak detection approach and best-fit model, as homozygous regions tend to have a unimodal distribution while heterozygous regions tend to have a bimodal distribution.

From the best-fit model, it is also possible to derive the homopolymer length value for both alleles in the homozygous (unimodal distribution) case, or two different homopolymer length values, one for each allele, in the heterozygous case (bimodal distribution). While this method may facilitate the identification of the length of short homopolymer regions as the associated flow space densities clearly exhibit a peak value, we observed that it is significantly more difficult to classify longer homopolymers as well as heteropolymers.

Pending patent application PCT/EP2019/065777 by Lin and Zu describes a genomic data analyzer configured to detect and characterize, with a variant calling module, genomic variants from next generation sequencing reads out of a pool of enriched genomic patient samples in repeat patterns regions of the human genome such as homopolymers or heteropolymers. The variant calling module may estimate the probability distribution of the length of the repeat pattern for each patient sample and cross-analyze it against other samples in a single experimental pool to identify best-fit variant models for each pair of samples. The variant calling module may further group samples according to their matching best-fit variant models and identify which group of patient samples carries the wild type reference without the need for control data in the pool.

The variant calling module may subsequently characterize the homozygous or heterozygous repeat patterns variants for each patient sample with improved specificity and accuracy even in the presence of next generation sequencing biases. This genomic data analyzer is however primarily suited to the case of variants for which a well characterized wild type reference (with constant length) can be assumed in the best-fit variant models.

### Microsatellite Status Detection with NGS

In the human genome, microsatellite sequences are commonly found as homopolymer (mononucleotide) or heteropolymer (several nucleotides, also known as short tandem repeats STR of 2 to 6 nucleotides) sequences, which may be repeated 5 to 50 times in the DNA sequences. More than 500000 microsatellite loci have been found in the human genome, in coding as well as non-coding regions, corresponding to near 3% of the human DNA. It is estimated that at least 100000 to 150000 of these loci are highly variable in the human population germline DNA.

Over 20 unstable microsatellite repeat loci have been identified as the cause of dozens of neurological diseases in human. Moreover, in somatic DNA mutations and in particular in tumor cells, microsatellite instability (MSI) is regularly observed as a genomic alteration due to insertions or deletions of a few nucleotides in the microsatellite repeat regions based upon one nucleotide repeat (homopolymers) or a few nucleotides (heteropolymers), due to a DNA mismatch repair system deficiency.

Thus, in a number of cancers, and in particular in uterine, colon and stomach cancers such as UCES (Uterine Corpus Endometrial Carcinoma), COAD (Colon Adenocarcinoma) and STAD (Stomach adenocarcinoma), a few microsatellite loci are routinely used as biomarkers in diagnosis and prognosis, for instance with the Bethesda panel, the Hamelin panel or the Promega test kit for Lynch syndrome.

Most commonly tested MSI biomarkers genomic loci are homopolymers of at least 20 bp such as BAT-25, a 25-repeat poly(T) tract located within intron 16 of the c-kit oncogene, which is typically found as a shorter version in tumor DNA; BAT-26, a 26-repeat poly(A) tract located within the fifth intron of the MSH2 gene, monomorphic at a length of 26 bp in 99% of individuals of ethnic European ancestry, yet polymorphic at a length of 15, 20, 22 or 23 bp in up to 25% of individuals of ethnic African ancestry, which is also typically found as a shorter version in tumor DNA; as well as NR-21, NR-22, NR-24, MONO-27, BAT-40, and/or CAT25.

More recently, MSI status has also been used to identify the most relevant personalized medicine treatment based on immunotherapy, for instance with pembrolizumab, and with immune checkpoint blockade therapy in solid tumors.

More generally, determining MSI status may be of interest to facilitate the diagnosis, choice of treatment and prognosis in a diversity of cancers such as colorectal adenocarcinoma, endometrial cancer, bladder cancer, breast carcinoma, cervical cancer, cholangiocarcinoma, esophageal and esophagogastric junction carcinoma, extrahepatic bile duct adenocarcinoma, gastric adenocarcinoma, gastrointestinal stromal tumors, glioblastoma, liver hepatocellular carcinoma, lymphoma, malignant solitary fibrous tumor of the pleura, melanoma, neuroendocrine tumors, NSCLC, female genital tract malignancy, ovarian surface epithelial carcinomas, pancreatic adenocarcinoma, prostatic adenocarcinoma, small intestinal malignancies, soft tissue tumors, thyroid carcinoma, uterine sarcoma, uveal melanoma, and any combination thereof.

Prior art MSI tests are primarily based upon PCR amplification of DNA fragments from tumor tissue samples, followed by capillary electrophoresis or melting curve analysis to measure the fragment length polymorphisms, and compared to the germline measurement from a blood sample to characterize the instability of each MSI loci length in the tumor test set relative to the patient germline MSI lengths. With those prior art non-NGS based panels, patients can be categorized as:

Microsatellite stable, MSS: no evidence of any of the biomarker loci exhibiting instability.

Microsatellite instable-low, MSI-L: evidence of instability in only one marker locus.

Microsatellite instable-high, MSI-H: evidence of instability in at least two marker loci.

These workflows do not scale well with the increasing medical demand for MSI characterization. Prior art MSI assays were primarily developed for colorectal cancer (CRC) and are associated with an increase in false negative in other tumors. In contrast, next-generation sequencing genomic analyzers facilitate the high throughput analysis of multiple genomic regions in multiplexed DNA samples, so that potentially many more MSI loci can be used as biomarkers with improved limits of detection (LOD), in particular with the development of circulating tumor DNA (ctDNA) and cell free DNA (cfDNA) NGS analysis methods. Moreover, beyond the 5 or 7 standard MSI loci tested in clinical oncology practice since the mid 2000s, recent research has identified many more MSI loci frequently mutated in tumor DNA and thus potentially associated with cancer diagnosis, prognosis, and treatment. As the cost of high throughput sequencing keeps on decreasing, there is a growing interest in extending the prior genomic data analytics, also on microsatellite variants.

WO2013/153130 by Lambrechts et al. from the VIB identifies a set of 106 frequent recurrently mutated genomic markers as homopolymers of 8 to 18 bp lengths (mostly in the 10-13 bp range) associated with the MMR-deficient tumors, which may be treated with PARP inhibitors, but they did not develop NGS experiments with these loci.

WO2017/112738 by Perry et al. from Myriad Genetics identifies another set of 35 homopolymer microsatellite loci which can be used to identify the MSI status of a tumor sample, by detecting 20 to 33 indels in the 35 microsatellite regions relative to a known reference DNA sequence or the sequence of germline DNA from the tested patient.

WO2018/037231 by Burn et al. from the University of Newcastle Upon Tyne discloses another set of 120 short markers which can discriminate between MSI-H and MSS status for colorectal cancer (CRC) and Lynch Syndrome diagnosis and chemotherapy treatment indication, for instance with pembrolizumab, irinotecan, bevacizumab, cisplatin, carboplatin, 5-fluorouracil (5-FU) and others. Burn et al. selected the markers specifically as short mononucleotide repeats of up to 12 nucleotides maximum, so that they are less likely to be polymorphic, and also explicitly excluded from their set those loci within this range which are known to be polymorphic in the human population. Thanks to the plausible exclusion of the polymorphic loci from the selected set of microsatellite sites, the patient tumor genomic data can be simply compared to the monomorphic wild type reference genome without the need to compare with the patient germline DNA as it would be required with polymorphic loci such as for instance BAT-26.

On the analytics side, the microsatellite regions remain particularly challenging to characterize with next-generation sequencing statistical bioinformatics methods because of the higher ratio of NGS biases in repeat regions, the low quality coverage signal in some NGS experiments, the low variant fraction in the input samples, and/or the MSI-specific fact that there is no strong germline wild type reference signal (with a predefined repeat loci length) to compare to for unbiasing purposes; indeed, many loci may be polymorphic and vary from patient to patient even in the germline cells.

As reviewed by Baudrin et al. in “Molecular and computational methods for the detection of microsatellite instability in cancer”, Frontiers in Oncology, December 2018 (https://doi.org/10.3389/fonc.2018.00621), several bioinformatics NGS methods have recently been developed in combination with different library preparation protocols (wet lab protocols, assays or kits), including Whole Genome Sequencing (WGS), Whole Exome Sequencing (WES) and targeted gene sequencing (TGS) protocols, the latter being either amplicon-based or capture-based.

Salipante et al. from the University of Washington proposed an NGS-based MSI detection method, called mSINGS, in “Microsatellite instability detection by next generation sequencing”, Clinical Chemistry 60:9, pp. 1192-1199 (2014), presenting NGS testing results for 15 to 2957 microsatellite markers out of a targeted gene enrichment. Their method compares the patient microsatellite lengths to those of a reference population of MSI-negative individuals, and provides a quantitative assessment of the MSI status over multiple loci rather than a qualitative MSS/MSI-L/MSI-H ternary classification.

The mSINGS method was later applied to a diversity of cancers by characterizing the MSI status across more than 200000 microsatellite loci, by using high throughput exome sequencing of tumor-normal tissue pairs, as part of the MOSAIC method described by Hause et al in “Classification and characterization of microsatellite instability across 18 cancer types”, Nature Medicine 22, 1342-1350 (2016).

Cortes-Ciriano et al. in “A molecular portrait of microsatellite instability across multiple cancers”, Nature Communications also evidenced the applicability of analyzing a broader set of microsatellite loci across the whole genome to characterize the MSI status in different cancers, using the comparisons of the distributions of the repeat lengths from tumour and matched normal genomes at each locus using the Kolmogorov-Smimof statistics.

As reviewed by Baudrin et al., the prior art methods MSISensor, MANTIS, MSings, the Cortes-Ciriano method and MSI-ColonCore use a similar genomic analysis workflow, comprising the steps of:
(i) read alignment of NGS raw reads data from tumor samples;
(ii) microsatellite loci identification (from 15 loci up to more than 1000 microsatellite markers);
(iii) indel/allele calling for each microsatellite locus;
(iv) for each locus, comparison of the repeat length distribution in the tumor samples with the reference repeat length distribution background model for normal samples (either baseline or paired reference depending on the genomic analysis tool) using statistical tests (Z-score, average distance, Kolomogorov-Smirnov or chi square depending on the genomic analysis tool); and
(v) for each tumor sample, MSI calling based on thresholding for a minimum ratio of instable loci out of the scores or distances calculated in the former steps for most tools, or, specifically for the Cortes-Ciriano method, a random forest analysis for the MSI status classification.

The FDA-approved FoundationOne CDx NGS panel from Foundation Medicine uses a customized analysis workflow designed to detect various genomic alterations, including microsatellite instability in 95 undisclosed intronic homopolymer repeat loci of length 10 to 20 bp. For each of these 95 MSI loci, the repeat length distribution for the sample is calculated over all mapping reads, and the mean and the variance of the repeat length distribution is used in a 190-dimension data projection into the MSI score, a single value corresponding to the first component of a principal component analysis. The MSI-H or MSS status is assessed by manual unsupervised clustering rather than by an automated workflow, which does not scale well with the increasing demand for NGS analytics by worldwide laboratory and hospitals.

Recent research by Foundation Medicine uses a similar approach, as reported for instance in “Real-time targeted genome profile analysis of pancreatic ductal adenocarcinomas identifies genetic alterations that might be targeted with existing drugs or used as biomarkers”, Gastrology 2019; 156:2242-2253 by Singhi et al. which reports the genomic MSI characterization of PCAD tumors from FFPE patient samples according to the NGS analysis of a subset of 114 intronic homopolymer loci out of a set of 1897 potential microsatellite loci with adequate coverage on FFPE samples on their custom targeted NGS workflow.

The 114 loci are filtered based on their hg19 reference repeat length which is chosen in the range of 10 to 20 bp so that, according to the authors, they are long enough to produce a high rate of DNA polymerase slippage but short enough to facilitate alignment in their NGS workflow with 49 bp read length. The measured mean and variance of the repeat length distributions of the NGS reads associated with each patient sample at each of the 114 loci are analyzed with principal component analysis to produce the MSI score, enabling to classify the tumor as MSS, MSI-ambiguous (but not necessarily MSI-L—the data is not discriminant enough for that classification), or MSI-H.

The FDA-approved MSK-Impact assay from the Memorial Sloan Kettering uses an NGS computational workflow with the MSIsensor tool from Niu et al. “MSIsensor: microsatellite instability detection using paired tumor-normal sequence data”, Bioinformatics 30(7):1015-1066 (2014). By comparing the DNA coverage for tumor samples with a panel targeting 468 genes associated with cancer against the matched normal DNA information, the MSK-Impact assay enables to assess the number and length of over 1000 microsatellite homopolymer markers rather than the limited subset of 5 or 7 MSI loci used by the prior art non-NGS assays.

The MSI loci are assumed somatic if the k-mer distributions are significantly different between the tumor and the matched normal genomic data as measured with a standard multiple testing correction of χ2 p-values. The tool calculates accordingly a continuous score rather than a discrete decision (MSS, MSI-L, MSI-H), and classifies the patient tumor as MSS if the score is below 10, as MSI-H otherwise. One major limitation of this assay, as well as of most prior art methods, is that it requires tumor/normal paired sequencing data; it cannot work with tumor-only data.

More recently, Niu at al. have announced an updated MSIsensor2 software working only with tumor sequencing data, indifferently from Cell-free DNA (cfDNA), Formalin Fixed Paraffin Embedded (FFPE) or other patient sample type, as announced at github.com/niu-lab/msisensor2. According to their benchmarks, the tool performs similarly to the MSIsensor software in terms of MSI classification, but it now runs 10 times faster on a targeted panel of 500 genes, as well as with WES or WGS data sets.

The authors mention that the tool is based on a novel machine learning algorithm, but did not disclose the source code or computational method details. They only indicated that for use with a dedicated MSI set, for instance on specific genes, the tool requires a dedicated training to build the data model specifically for the selected MSI set. No constraints on the choice of the MSI loci has been disclosed by the authors, but, according to the github file updates on Oct. 12, 2019, the early version of the software was using the same parametrization options as MSIsensor, assuming a default minimal homopolymer size of 5 and a maximal size of 50 base pairs, and a default minimal homopolymer size for distribution analysis of 10. With the MSIsensor tool, these parameters were configurable as command line options, while in the latest MSIsensor2 release, they are predefined to default values and may actually vary with the machine learning model, possibly throughout the whole MSI.

WO2019/108807 by Georgiadis et al. from Personal Genome Diagnostics describes a process for reporting an MSI status from a cell-free DNA sample of the patient blood or plasma in order to determine whether the patient cancer is suitable for immune checkpoint inhibitor therapy comprising an antibody such as anti-PD-1, anti-IDO, anti-CTLA-4, anti-PD-L1 or anti-LAG-3. The process comprises comparing how the peaks of the length distribution of the measured repeat tract deviate from peaks of a reference repeat length distribution from a matched normal DNA sample corresponding to the healthy model, after some barcoding error correction steps to facilitate the digital peak finding (DPF). The proposed DPF method retains only the clearly discriminating local peaks with sufficient coverage once filtered based on several simple local heuristics. The method operates either on standard MSI subsets of 5 loci (BAT25, BAT26, MONO27, NR21, NR24) or 7 loci (BAT-25, BAT-26, MONO-27, NR-21, NR-24 homopolymers, and PentaC and PentaD heteropolymers), or with an additional set of 65 undisclosed microsatellite regions. Samples are classified as MSI-H if >20% of loci were MSI. This process assumes the availability of a reference repeat length distribution from a matched normal DNA sample, and therefore cannot work in a workflow using solely a tumor sample.

WO2020002621 by Hoffmann La Roche and Roche Diagnostics proposes a method of detecting the MSI status out from up to 170 loci comprising a short tandem repeat (STR, that may be homopolymer repeats of a single nucleotide or heteropolymer repeats of a pattern of 2 to 6 nucleotides). For each STR locus, a t-statistic metric is calculated as a function of the mean and the variance of the sample repeat length distribution (RLD) on the one hand and from the mean and the variance of a background RLD model on the other hand. The resulting RLD metric is then individually compared to an independent threshold value at each locus, and the MSI status is derived from quantifying the number of loci for which the RLD metric exceeds the local threshold. In this method, no tumor-normal matching is required, but instead the MSI detection method uses a pre-computed “normal” repeat length distribution, which is pre-established offline according to a number of constraining requirements. In particular the method requires to exclude MSI loci that cannot be fit into a clear single-peak RLD distribution background model (so that the t-statistic metric based on mean and variance is suitable). This requires excluding microsatellite loci with germline variants in certain human populations, which may hinder in practice the large-scale use of this method in a high-throughput genomic data analysis system across large pools of patients of diverse ethnic backgrounds.

US2020/032332 by Guangzhou Burning Rock DX describes the prettyMSI method using a multi-gene targeted capture detection of 15 to 22 microsatellite loci corresponding to homopolymers of 11 to 27 nucleotide long. The stability status of each locus is estimated based on the target peak height ratio in a next generation sequencing experiment using only tumor tissue. Similar to the Roche method, this method assumes that there is a significant peak difference between a reference repeat length distribution background model for the normal value at a MSI locus and its somatic counterpart, and uses up to 22 microsatellite loci for which this assumption enables to discriminate the MSI status of colorectal tumor samples based on a simple local peak measurement method, here again using the mean and standard deviation of the repeat length distribution at each loci. When there is more than one peak in the sample repeat length distribution, a heuristic is proposed to only consider the length type corresponding to the highest peaks-thus possibly mis-measuring certain MSI events.

There is, therefore, a need for improved high throughput genomic analysis methods to characterize the MSI status of patient samples that jointly meet the following requirements: the methods should be suitable for an automated NGS workflow not requiring manual classification, and fast enough to be deployed at a large scale in current computational architectures and across a diversity of populations of different genetic origins; the methods should provide comparable sensitivity and specificity compared to the current clinical practice MSI testing protocols and assays, while being indifferently applicable to various sets of microsatellite loci in accordance with the actual clinical application needs; the methods should not rely upon somatic-normal pair matching with a germline sample; still, the methods should be suitable for detection even with low somatic variant fraction below 10% of the sample, for instance out of FFPE samples or liquid biopsy samples; and in particular the methods should not rely on the assumption that the repeat length obtained for a given somatic sample is similarly distributed as the data obtained from reference samples with normal (MSS) status.

### SUMMARY OF THE INVENTION

A method is accordingly proposed for determining a microsatellite instability (MSI) status of a patient comprising: obtaining DNA fragments from a biological DNA sample from a patient, the sample comprising cells from a solid tissue or a bodily fluid; sequencing the DNA fragments with a high throughput sequencing technology to obtain a plurality of data reads for each DNA fragment; aligning with a computer processor the data reads to a reference genome DNA sequence comprising a predefined set of N microsatellite genomic loci; at each microsatellite genomic locus i in the set of microsatellite genomic loci, measuring with the computer processor a patient sample distribution D_{MSI} of the nucleotide repeat lengths for the set of aligned data reads mapped to the reference genome DNA sequence at the microsatellite locus i and estimating a local MSI status score sᵢ as a function of the difference of the measured patient sample distribution D_{MSI} of the nucleotide repeat lengths relative to a reference background distribution model D_{MSS} of the nucleotide repeat lengths at the microsatellite genomic locus i; determining a global MSI score S to characterize the MSI status of the patient sample as a function of all estimated local MSI status scores sᵢ, i=1 to N, each microsatellite genomic locus in the predefined set being a STR. In a possible embodiment, the STR may be a mono-homopolymer repeat of a single nucleotide and having a reference repeat length of at least 13 nucleotides (13 bp) and at most 25 nucleotides (25 bp), but other embodiments are possible.

In a possible embodiment, the local MSI status score sᵢ may be calculated as a function of at least two independent scalar parameters p₁, p₂ of a multi-parametric function F(D_{MSS}, p₁, p₂) of the reference background distribution model D_{MSS} of the nucleotide repeat length such that D_{MSI}=F(D_{MSS}, p₁, p₂) at the microsatellite genomic locus i.

A method is also proposed for determining a microsatellite instability (MSI) status of a patient comprising:
(i) obtaining DNA fragments from a biological DNA sample from a patient, the sample comprising cells from a solid tissue or a bodily fluid;
(ii) sequencing the DNA fragments with a high throughput sequencing technology to obtain a plurality of data reads for each DNA fragment; and
(iii) aligning the data reads to a reference genome DNA sequence comprising a predefined set of N microsatellite genomic loci;
the method further comprising:
(iv) for each microsatellite genomic locus i in the predefined set of microsatellite genomic loci, obtaining a reference repeat length distribution D_{MSS} of the nucleotide repeat length at the microsatellite genomic locus i;
(v) determining repeat length distribution D_{MSI} of the nucleotide repeat lengths from the set of aligned data reads from the patient sample mapped to the reference genome DNA sequence at the microsatellite locus i;
(vi) characterized in that the method further comprises
(vii) estimating, with a curve-fitting method, at least two independent scalar parameters p1, p2 of a multi-parametric function F(D_{MSS}, p1, p2) of the reference background repeat length distribution model D_{MSS} such that the measured patient sample repeat length distribution D_{MSI}=F(D_{MSS}, p1, p2) at the microsatellite genomic locus i;
(viii) estimating a local MSI score as a function of the at least two independent scalar parameters p1, p2 at the microsatellite genomic locus i;
(ix) determining a global MSI score S to characterize the MSI status of the patient sample as a function of all estimated local MSI scores s¹, i=1 to N; and
(x) determining the microsatellite instability (MSI) status of the patient as positive if the global MSI score S over the N loci is above a predefined cutoff value.

In a further possible embodiment, the local MSI status score sᵢ may be calculated as a function of three independent scalar parameters p₁, p₂, p₃ of a multi-parametric function F(D_{MSS}, p₁, p₂, p₃) of the reference background distribution model D_{MSS} of the nucleotide repeat length distribution D_{MSS} such that D_{MSI}=F(D_{MSS}, p₁, p₂, p₃) at the microsatellite genomic locus i.

The independent scalar parameters may be the variant fraction p₁ corresponding to the ratio of somatic DNA content relative to the total DNA content in the patient sample, a microsatellite length shift value p2 characterizing by how many repeat insertions or deletions the somatic microsatellite length has shifted in the somatic DNA normalized relative to the microsatellite stable status length at the microsatellite genomic locus I, and possibly a microsatellite length stability value p3 characterizing how variable the somatic microsatellite length shift is in the somatic DNA content.

The independent scalar parameters p={p₁, p₂} or p={p₁, p₂, p₃} at locus i may be inferred by minimizing the difference between the measured patient repeat length distribution D_{MSI} and the predicted patient repeat length distribution F(D_{MSS}, p) at locus i.

The local MSI status score may be estimated as a function of at least two of the independent scalar parameters at each locus i, and the global MSI score may be calculated as the sum of the local MSI status scores over the N loci, or a normalized MSI score calculated as the sum of the local MSI status scores normalized to the highest local MSI score over the N loci, and/or an MSI score count calculated as the number of loci in the set of N loci where the local MSI score is over a predefined threshold.

The microsatellite instability (MSI) status of the patient may be determined as a positive status if the global MSI score S over the N loci is above a predefined cutoff value, and as a negative status if the global MSI score S over the N loci is below the predefined cutoff value.

The microsatellite instability (MSI) status of the patient may be determined as a negative status if the global MSI score S over the N loci is below a predefined cutoff value.

Each microsatellite genomic locus in the predefined set may be a homopolymer repeat of a single nucleotide.

Each microsatellite genomic locus in the predefined set may have a reference repeat length of at least 13 nucleotides (13 bp) and at most 25 nucleotides (25 bp).

In some aspects, a method for determining a microsatellite instability score of a subject is envisioned, the method comprising:
a. obtaining nucleic acids from the subject;
b. preparing a plurality of nucleic acid fragments for high-throughput sequencing;
c. subjecting the prepared nucleic acid fragments to high-throughput sequencing;
d. cleaning and aligning, with a computer processor, the sequencing reads to a reference genome;
e. identifying, with a computer processor, the reads spanning at least one MSI locus;
f. computing, with a computer processor, a MSI score for each of the at least one MSI loci by performing the steps of:
   i. recording the repeat length of each read spanning the MSI locus;
   ii. establishing a repeat length distribution D_{MSI} based on the reads spanning the MSI locus;
   iii. comparing the repeat length distribution D_{MSI} to a reference repeat length distribution D_{MSS}, wherein the comparing involves optimizing a model with at least three parameters that represent the biological events underlying MSI;
   iv. computing a MSI score based on at least two of the optimized parameters;
g. aggregating the MSI scores across the at least one MSI loci to obtain a global MSI score; and
h. reporting the global MSI score to the user.

In some embodiments, the nucleic acid is genomic DNA or cell-free DNA extracted from the subject, wherein the subject is suffering from, or suspected to suffer from, cancer.

In some embodiments, the nucleic acid contains a mixture of cancerous cell DNA and normal cell DNA.

In some embodiments, comparing the repeat length distribution D_{MSI} to a reference repeat length distribution D_{MSS} involves estimating, with a computer processor, using a curve-fitting method, three independent scalars of a multi-parametric function.

In some embodiments, the three scalars represent respectively the shift in microsatellite length in cancerous cells compared to the reference, the variability of microsatellite lengths in cancerous cells compared to the reference, and the fraction of the sample that represents cancerous cells as opposed to normal cells.

In some embodiments, preparing the plurality of nucleic acid fragments for high-throughput sequencing involves ligating adaptors to end-repaired DNA fragments.

In some embodiments, endogenous molecular identifiers are used to assign reads to read families encompassing potential PCR duplicates.

In some embodiments, the adapters contain exogenous molecular identifiers.

In some embodiments, the exogenous molecular identifiers are used in combination with endogenous molecular identifiers to assign reads to read families encompassing potential PCR duplicates.

In some embodiments, establishing a repeat length distribution D_{MSI} based on the reads spanning the MSI locus involves computing the one or more most likely repeat lengths for each read family and computing D_{MSI} as the distribution of family-level repeat lengths.

In some embodiments, the global MSI score is transformed into a MSI status based on at least one threshold.

A computer system to implement the estimation of the MSI scores from methods described herein is also envisioned.

A genomic data analyzer incorporating the computer system of described herein is also envisioned, wherein the genomic data analyzer also performs the cleaning and aligning of the sequencing reads to a reference genome and the identifying of the reads spanning at least one MSI locus.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 is a schematic representation of a genomic analysis workflow comprising a laboratory process (also known as the “wet lab” process) and a bioinformatics workflow (also known as the “dry lab” process).

FIG. 2 is a schematic representation of an exemplary MSI analysis computational workflow according to some embodiments of the present disclosure.

FIG. 3 is a schematic representation of an exemplary workflow to compute the repeat length distribution taking into account read family information.

FIGS. 4A-4H illustrate the theoretical transformation of the length distribution of a background stable model into different possible MSI lengths distribution according to different variables, namely the variant fraction (p₁), the average somatic MSI length shift relative to a reference background stable model distribution length peak (p₂) and the variability of the MSI length (p₃) into samples comprising different variant fractions of somatic DNA over total DNA. FIG. 4A illustrates an exemplary contribution of different p₁, p₂ and p₃ values to the transformed distribution F(D_{MSS}), specifically, if p₂ equals to 0, even if p₁ is 100%, the transformed distribution will be the same as original. FIG. 4B illustrates an exemplary contribution of different p₁, p₂ and p₃ values to the transformed distribution F(D_{MSS}), specifically, if p₁ equals to 0, even if p₂ is 100%, the transformed distribution will be the same as original. FIG. 4C illustrates an exemplary contribution of different p₁, p₂ and p₃ values to the transformed distribution F(D_{MSS}), specifically, with a somatic variant fraction at 100% (p₁=1) and a deletion of 8 bp (p₂=0.5 on germline reference of 16 bp) the whole MSS distribution is shifted by 0.5*16=8 bps to the left. FIG. 4D illustrates an exemplary contribution of different p₁, p₂ and p₃ values to the transformed distribution F(D_{MSS}), specifically, with a somatic variant fraction at 100% (p₁=1) and a deletion of 1 bp (p₂=0.0625 on the germline reference of 16 bp) the whole MSS distribution is shifted 0.0625*16=1 bp to the left. FIG. 4E illustrates an exemplary contribution of different p₁, p₂ and p₃ values to the transformed distribution F(D_{MSS}), specifically, with a somatic variant fraction at 50% (p₁=0.5), half of the MSS distribution is shifted by 0.5*16=8 bps (p₂=0.5 on the germline reference of 16 bp) to the left, and half of the MSS distribution remains unchanged. FIG. 4F illustrates an exemplary contribution of different p₁, p₂ and p₃ values to the transformed distribution F(D_{MSS}), specifically, with a somatic variant fraction at 50%, (p₁=0.5), half of the MSS distribution is shifted 0.0625*16=1 bp to the left (p₂=0.0625 on the germline reference of 16 bp), and half of the MSS distribution remains unchanged. FIG. 4G illustrates an exemplary contribution of different p₁, p₂ and p₃ values to the transformed distribution F(D_{MSS}), specifically, with a somatic variant fraction at 50% (p₁=0.5), half of the MSS distribution is shifted by 0.5*16=8 bps (p₂=0.5 on the background reference of 16 bp) to the left, and half of the MSS distribution remains unchanged (similar to FIG. 4E). The somatic peak attenuation parameter p₃=0.5 instead of 1 changes the shape of the somatic peak as half shorter but twice wider than the left somatic peak of FIG. 4E), while the background reference peak (right small peak in FIG. 4E) will remain unchanged. FIG. 4H illustrates an exemplary contribution of different p₁, p₂ and p₃ values to the transformed distribution F(D_{MSS}), specifically, the somatic peak attenuation parameter p₃=0.25 instead of 1 changes the shape of the somatic peak as one quarter shorter but four times wider, while the germline peak remains unchanged.

FIG. 5 represents an exemplary microsatellite repeat lengths distribution for a background stable model (MSS), a measured patient sample (MSI), and the fitted MSI repeat length distribution (MSI-fitting) and its inferred parametrization (p₁, p₂, and p₃) according to the proposed methods, at one microsatellite locus. This example indicates a very limited shift in the microsatellite length in the patient sample (p₂ is close to 0).

FIG. 6 represents an exemplary microsatellite repeat lengths distribution for a background stable model (MSS), a measured patient sample (MSI), and the fitted MSI repeat length distribution (MSI-fitting) and its inferred parametrization (p₁, p₂, and p₃) according to the proposed methods, at one microsatellite locus. This example supports a modest shift in microsatellite length (p₂), with a medium variant fraction (p₁)

FIG. 7 represents an exemplary microsatellite repeat lengths distribution for a background stable model (MSS), a measured patient sample (MSI), and the fitted MSI repeat length distribution (MSI-fitting) and its inferred parametrization (p₁, p₂, and p₃) according to the proposed methods, at one microsatellite locus. This example support a marked shift in the microsatellite length (p₂) with a variant fraction close to 0.5 (p₁).

FIG. 8 illustrates an embodiment of an environment in which the systems and methods of the present disclosure may be practiced.

FIG. 9 illustrates an embodiment of a block diagram of an electronic device.

### DETAILED DESCRIPTION OF THE INVENTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

The particulars shown herein are by way of example and for purposes of illustrative discussion of the various embodiments only and are presented in the cause of providing what is believed to be the most useful and readily understood description of the principles and conceptual aspects of the methods and compositions described herein. In this regard, no attempt is made to show more detail than is necessary for a fundamental understanding, the description making apparent to those skilled in the art how the several forms may be embodied in practice.

The proposed methods and systems will now be described by reference to more detailed embodiments. The proposed methods and systems may, however, be embodied in different forms and should not be construed as limited to the embodiments set forth herein. Rather, these embodiments are provided so that this disclosure will be thorough and complete, and will fully convey the scope to those skilled in the art.

Unless otherwise defined, all technical and scientific terms used herein have the same meaning as commonly understood by one of ordinary skill in the art to which this invention belongs. The terminology used in the description herein is for describing particular embodiments only and is not intended to be limiting. As used in the description and the appended claims, the singular forms “a,” “an,” and “the” are intended to include the plural forms as well, unless the context clearly indicates otherwise.

Unless indicated to the contrary, the numerical parameters set forth in the following specification and attached claims are approximations that may vary depending upon the desired properties sought to be obtained and thus may be modified by the term “about”. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

### Definitions

As used herein, an “adapter” or “adaptor” refers to a short double-stranded or partially double-stranded DNA molecule that has been designed to be added to a DNA fragment. An adaptor may have blunt ends, sticky ends as a 3′ or a 5′ overhang, or a combination thereof. For example, to improve ligation efficiency, an adenine may be added to each of the 3′ blunt ends of the fragmented DNA prior to adaptor ligation, and the adaptor may have a thymidine overhang on the 3′ end to base-pair with the adenine added to the 3′ end of the fragmented DNA. The adaptor may have a phosphorothioate bond before the terminal thymidine on the 3′ end to prevent an exonuclease from trimming the thymidine, thus creating a blunt end when the end of the adaptor being ligated is double-stranded.

As used herein, the terms “aligning” or “alignment” or “aligner” refer to mapping and aligning base-by-base, in a bioinformatics workflow, the pre-processed sequencing reads to a reference genome sequence, which may vary among applications. For instance, in a targeted enrichment application where the sequencing reads are expected to map to a specific targeted genomic region in accordance with the hybrid capture probes used in the experimental amplification process, the alignment may be specifically searched relative to the corresponding sequence, defined by genomic coordinates such as the chromosome number, the start position and the end position in a reference genome. The alignment may however also be performed against the whole reference genome, with subsequent filtering out of reads mapping to regions not targeted by the capture panel. The alignment may include a local refinement of the initial alignment to match insertions and deletions inferred within a given genomic window.

As used herein, the term “amplification” refers to a polynucleotide amplification reaction to produce multiple polynucleotide sequences replicated from one or more parent sequences. Amplification may be produced by various methods, for instance a polymerase chain reaction (PCR), a linear polymerase chain reaction, a nucleic acid sequence-based amplification, a rolling circle amplification, and other methods.

As used herein, the terms “cell-free DNA” and “cfDNA” refer to DNA molecules that circulate in a subject's body and originate from one or more healthy cells and/or from one or more cancer cells. These DNA molecules are found outside cells, in bodily fluids such as blood, whole blood, plasma, serum, urine, cerebrospinal fluid, fecal, saliva, sweat, tears, pleural fluid, pericardial fluid, or peritoneal fluid of a subject, and are fragments of DNA expelled from healthy and/or cancerous cells, e.g., upon apoptosis and lysis of the cellular envelope. The cell-free DNA can be in the form of microvesicles, exosomes, apoptotic bodies, or DNA-protein complexes. The fraction of cfDNA that originates from cancerous cells is sometimes referred to as “circulating tumor DNA” or “ctDNA”.

As used herein, the terms “complementary DNA” or “cDNA” refer to a synthetic DNA reverse transcribed from RNA through the action of a reverse transcriptase. The cDNA may be single stranded or double stranded and can include strands that have either or both of a sequence that is substantially identical to a part of the RNA sequence or a complement to a part of the RNA sequence.

As used herein, the term “consensus sequencing” refers, in a bioinformatics workflow, to grouping sequencing reads into families of reads that may be issued from the same double-stranded DNA fragment and/or the same DNA fragment strand and generating a single sequence, called consensus sequence, that represents the group of reads. The consensus sequence may represent the most frequent sequence among the reads in the family or may be composed, at each position, of the most frequent nucleotide at the position among reads of the family. Other consensus-building methods are possible. Variant calling is then performed by processing the resulting consensus sequences, rather than the totality of reads.

As used herein, the term “coverage” in reference to NGS refers to the number of reads that align to, or “cover” known reference bases. The coverage may be reported per position or as the average or median over multiple positions. The sequencing coverage level determines whether variant discovery can be made with a certain degree of confidence at particular base positions. Across multiple genomic regions, the average coverage can be computed as the read count, often considering only high-quality reads effectively mapped onto the reference genome, multiplied by the read length and divided by the total length of the considered genomic regions. At a higher level of coverage, each base is covered by a greater number of aligned sequence reads, and mutations compared to a reference sample can be determined.

As used herein, a “DNA sample” refers to a nucleic acid sample derived from an organism, as may be extracted for instance from a body tissue or fluid. The organism may be a human, an animal, a plant, fungi, or a microorganism. The nucleic acids may be found in limited quantity or low concentration, such as fetal circulating DNA (cfDNA) or circulating tumor DNA in blood or plasma. A DNA sample also applies herein to describe RNA samples that were reverse-transcribed and converted to cDNA.

As used herein, a “DNA fragment” refers to a short piece of DNA resulting from the fragmentation of high molecular weight DNA. Fragmentation may have occurred naturally in the sample organism, or may have been produced artificially from a DNA fragmenting method applied to a DNA sample, for instance by mechanical shearing, sonification, enzymatic fragmentation and other methods. After fragmentation, the DNA pieces may be end repaired to ensure that each molecule possesses blunt ends. To improve ligation efficiency, an adenine may be added to each of the 3′ blunt ends of the fragmented DNA, enabling DNA fragments to be ligated to adaptors with complementary dT-overhangs.

As used herein, a “DNA product” refers to an engineered piece of DNA resulting from manipulating, extending, ligating, duplicating, amplifying, copying, editing and/or cutting a DNA fragment to adapt it to a next-generation sequencing platform.

As used herein, a “DNA-adaptor product” refers to a DNA product resulting from ligating a DNA fragment with a DNA adaptor to adapt it to a next-generation sequencing workflow. The DNA adaptor may contain one or more primer-binding sites, one or more sample barcodes (also known as indexes), one or more molecular identifiers, or other sequences useful for downstream analyses.

As used herein, a “DNA library” refers to a collection of DNA products or DNA-adaptor products to adapt DNA fragments for compatibility with a next-generation sequencing platform.

The term “enrich” as used herein refers to increasing the proportion of a desired substance, for example, to increase the relative frequency of at least one nucleic acid sequence compared to its natural frequency in a high throughput sequencing reaction. Positive selection, negative selection, or both are generally considered necessary to any enrichment scheme. Enrichment methods include, without limitation, hybrid-capture enrichment and amplification-based enrichment.

As used herein, the term “exon” refers to the continuous segment of a gene that will form a part of the final mature messenger RNA (mRNA) produced by that gene after introns have been removed by RNA splicing and that will be translated into part of the protein.

As used herein, the terms “genomic alteration,” “mutation,” and “variant” refer to a detectable change in the genetic material of one or more cells. A genomic alteration, mutation, or variant can refer to various types of changes in the genetic material of a cell, including changes in the primary genome sequence at single or multiple nucleotide positions, e.g., a single nucleotide variant (SNV), a multi-nucleotide variant (MNV), an indel (e.g., an insertion or deletion of nucleotides), a DNA rearrangement (e.g., an inversion or translocation of a portion of a chromosome or chromosomes), a variation in the copy number of a locus (e.g., a loss or a gain of an exon, gene, or a large span of a chromosome; also called copy number variation; “CNV”), a partial or complete change in the ploidy of the cell, a gene fusion (e.g., a rearrangement leading to parts of two different genes being fused to create a single chimeric gene), a variation in the expression level of a gene, as well as changes in the epigenetic information of a genome, such as altered DNA methylation patterns.

As used herein, the term “Genomic Data Analyzer” refers to a system using at least one computer processor to perform analyses of genomic data, also known as bioinformatic analyses. A genomic data analyzer may take as input files sequencing data, in formats such as binary base call (BCL) or FASTQ. The genomic data analyzer may return lists of variants, various genomic scores, and/or annotation in downloadable files, such as txt, pdf or spreadsheets. The genomic data analyzer may also return results in a user interface or may interact with other software to transfer results. A single genomic data analyzer may be responsible for the entire bioinformatic workflow, but in other settings different genomic data analyzer may be responsible for different parts of the bioinformatic workflow. The genomic data analyzer may be comprised of multiple scripts and software programs, some of which may be custom designed, or the genomic data analyzer may be a stand-alone software. The genomic data analyzer may be locally installed on the user computer or the genomic data analyzer may be cloud based and accessible using a dedicated software or a web browser.

As used herein, the term “homopolymer” refers to a nucleotide sequence consisting of a single, repeating nucleotide base (e.g., AAAAA or TTTTT) rather than a mixed sequence.

The term “hybridization” refers to the process of combining complementary, single-stranded nucleic acids into a single double-stranded molecule. Nucleotides will bind to their complement under normal conditions, so two perfectly complementary strands will bind (or ‘anneal’) to each other readily. Nucleotide inconsistencies between the two strands make binding between them more energetically unfavorable. However, hybridization can occur despite inconsistencies and the number of nucleotide differences tolerated for successful hybridization depends on the length of the DNA segment, the GC content of the DNA segment the temperature of the reaction, the salt concentration in the liquid, and the pH of the solution.

The term “intron” as used herein refers to any nucleotide sequence within a gene that is not expressed or operative in the final mRNA product. When present, introns separate exons within genes. Intron sequences are typically spliced out during the maturation of mRNA.

As used herein, the term “ligation” refers to the joining of separate DNA sequences, which may be single-stranded, double-stranded, or partially double-stranded. When double-stranded, the initial DNA molecules may be blunt ended or may have compatible overhangs to facilitate their ligation. Ligation may be produced by various methods, for instance using a ligase enzyme, performing chemical ligation, and other methods.

As used herein, the term “Microsatellite” refers to the multiple, continuous repetition of nucleotide patterns of one to nine base pairs, typically 5 to 50 times, in a genomic sequence. The human genome hosts hundreds of thousands of microsatellite loci, and microsatellites are prone to indel mutations (nucleotide insertions, deletions and combination thereof). Out of those, polymorphic microsatellites are microsatellites found with a high variability among a human population, with typically more than 1% heterozygosity for the microsatellite repeat length found in healthy individuals germline data.

As used herein, the term “microsatellite instability” or “MSI” refers to a genomic hypermutability condition that is observed via alterations in the length of one or more microsatellite repeats, possibly due to a deficiency in the MMR (DNA mismatch repair) pathway. MSI has been associated with a number of cancers, such as, but not limited to, colorectal, gastric and endometrial cancers.

As used herein, the term “microsatellite stable” or “MSS” refers to the stable, normal status of microsatellites in a subject.

As used herein, the term “molecular tag” or “molecular barcode” or “molecular code” or “molecular identifier” refers to a molecular arrangement such as a nucleic acid sequence, which is fully and uniquely specified by its string of nucleotides. Molecular identifiers can be endogenous, as represented by the position of the nucleic acid sequence with respect to a reference genome, the start and/or end sequences of the DNA nucleic acid sequence, or other properties of the nucleic acid sequences. Molecular identifiers can be exogenous, based on sequences incorporated during the library preparation. Exogenous molecular identifiers may be incorporated as part of a ligated adaptor or as part of primers used for amplification. Exogenous molecular identifiers may consist in a large pool of distinct sequences or a limited number of distinct sequences. Exogenous molecular identifiers may differ from each other based on their nucleotide sequence, their length, or a combination of sequence and length. Unique molecular identifiers can result from endogenous molecular identifiers, exogenous molecular identifiers, or a combination of endogenous and exogenous molecular identifiers, as described in Kivioja et al. 2012, “Counting absolute numbers of molecules using unique molecular identifiers”, Nature Methods 9, 72-74. Unique molecular identifiers can be used to identify reads potentially representing PCR duplicates originating from the same original DNA fragment.

As used herein, the terms “Next Generation Sequencing” or “NGS” or “High-Throughput Sequencing” refer to a method of parallel sequencing. For instance, a nucleic acid (e.g., DNA) sample is obtained and prepared into a library (meaning a collection of nucleic acid fragments from the sample). The library may be prepared by fragmenting the DNA or RNA sample. Fragmentation can be performed by physical (e.g., sheared by acoustics, nebulization, centrifugal force, needles, or hydrodynamics) or enzymatic (e.g., site-specific or non-specific nucleases) methods. According to some embodiments, the fragments are about 200 bp, about 250 bp, about 300 bp, or about 350 bp in length. The DNA or RNA samples are repaired at the ends (e.g., blunt-ended) and then A-tailed (e.g., an adenosine is added to the 3′ end resulting in an overhang). Adapters are ligated to each end. The term “NGS read length” as used herein refers to the number of base pairs (bp) sequenced from a DNA fragment, or each end of a DNA fragment. After sequencing, the sequencing reads may be aligned onto a reference genome, enabling comparison between the sample and the reference.

A “nucleotide sequence” or a “polynucleotide sequence” refers to any polymer or oligomer of nucleotides such as cytosine (represented by the C letter in the sequence string), thymine (represented by the T letter in the sequence string), adenine (represented by the A letter in the sequence string), guanine (represented by the G letter in the sequence string) and uracil (represented by the U letter in the sequence string). It may be DNA or RNA, or a combination thereof. It may be found permanently or temporarily in a single-stranded or a double-stranded shape. Unless otherwise indicated, nucleic acids sequences are written left to right in 5′ to 3′ orientation.

As used herein, a “PCR duplicate” refers to a copy generated by PCR amplification from a single stranded DNA molecule belonging to an original DNA fragment or a DNA-adaptor product derived from an original DNA fragment.

As used herein, the term “pool” refers to multiple DNA samples (for instance, 8, 16, 48 samples, 96 samples, or more) derived from the same or different organisms, as may be multiplexed into a single high-throughput sequencing analysis. Each sample may be identified in the pool by a unique sample barcode.

As used herein, the term “primer sequence” refers to a nucleotide sequence comprising a region of complementarity to a target DNA a part or all of which is to be elongated or amplified.

As used herein, the term “probabilistic sequencing” refers, in a bioinformatics workflow, to grouping sequencing reads into families of reads issued from the same double-stranded DNA fragment and/or the same DNA fragment strand and performing variant calling directly on this data, by processing the totality of reads from different families in order to compute the statistical support for all possible genotypes at each genomic position to be analyzed. The statistical support may then be used for variant calling, by identifying genotypes with a support exceeding the level expected in the absence of variant according to a statistical model.

As used herein, the term “proliferation” refers to the increase in cell number through the combined processes of cell growth and division. It is a tightly regulated, fundamental biological process necessary for tissue development, maintenance, and wound healing. The cycle involves growth (G1, G2 phases), DNA synthesis (S phase), and division (M phase), often controlled by checkpoints.

As used herein, “read trimming” or “read pre-processing” refers, in a bioinformatics workflow, to the filtering out, in the sequencing reads, of a set of nucleotides at the start of the read sequence string, such as for instance the nucleotides corresponding to the adaptor sequences, to extract the real DNA fragment sequence to be analyzed. Read pre-processing may include determining the unique molecular identifiers of the sequencing reads.

As used herein, the term “reverse transcription” or grammatical variations thereof, refers to the process of copying the nucleotide sequence of an RNA molecule into a DNA molecule. Reverse transcription can be done by reacting an RNA template with an RNA-dependent DNA polymerase (also known as a reverse transcriptase) under well-known conditions. A reverse transcriptase is a DNA polymerase that transcribes single-stranded RNA into single stranded DNA. Depending on the polymerase used, the reverse transcriptase can also have RNase H activity for subsequent degradation of the RNA template.

As used herein, the term “sample barcode” or “sample index” or “index” is a known nucleotide sequence inserted in the DNA products during the library preparation to differently mark DNA fragments originating from different samples. The sample barcodes may be inserted as part of the ligated adaptors or may be subsequently incorporated as part of PCR primers. The sample barcodes are selected to differ among samples processed as part of the same pool, so that barcodes can be used to recognize and separate the reads corresponding to each sample as part of a step known as demultiplexing. Sample barcodes are often designed to not vary among reads originating from the same samples, but some systems include diverse indexes by sample, which do not overlap with those allocated to other samples.

As used herein, the term “sequencing” refers to reading a sequence of nucleotides as a string. High throughput sequencing (HTS) or next-generation-sequencing (NGS) refers to real time sequencing of multiple sequences in parallel, typically between 50 and a few thousand base pairs. Exemplary NGS technologies include those from Illumina, Ion Torrent Systems, Oxford Nanopore Technologies, Complete Genomics, Pacific Biosciences, and others. Depending on the actual technology, NGS sequencing may require sample preparation with sequencing adaptors or primers to facilitate further sequencing steps, as well as amplification steps so that multiple instances of a single parent molecule are sequenced, for instance with PCR amplification prior to delivery to flow cell in the case of sequencing by synthesis.

As used herein, the term “sequence reads” or “reads” refers to nucleotide sequences produced by any nucleic acid sequencing process described herein or known in the art. Reads can be generated from one end of nucleic acid fragments (“single-end reads”) or from both ends of nucleic acid fragments (e.g., paired-end reads, double-end reads). The length of the sequence read is often associated with the particular sequencing technology. High-throughput methods, for example, provide sequence reads that can vary in size from tens to thousands of base pairs (bp).

As used herein, the term “short tandem repeat” or “STR” refers to nucleotide sequences repeated in succession, forming highly polymorphic heteropolymers. They consist of repeating units of nucleotides, which typically span between one and six different nucleotides, although longer units are possible. For example, some STRs consist of repeating units of dinucleotides (e.g., ATATAT), or trinucleotides (e.g., CAGCAG), but may comprise longer repeating units.

As used herein, the term “variant calling” or “variant caller” or “variant call” refers to identifying, in the bioinformatics workflow, actual variants in the aligned reads. Variants are typically defined as difference with respect to the reference genome used. Variants may include single nucleotide permutations (SNPs; also known as single nucleotide variants, SNVs), insertions or deletions (INDELs), copy number variants (CNVs), as well as large rearrangements, substitutions, duplications, translocations, and others. Preferably variant calling is robust enough to sort out the real variants from the amplification and sequencing noise artefacts.

### Genomic Analysis Workflow

The methods disclosed may be integrated indifferently into a diversity of NGS genomic data analysis wet lab workflow systems. In some embodiments, tumor sample DNA may be extracted from a formalin fixed paraffin embedded (FFPE) sample. In some embodiments, the tumor sample DNA may be extracted from a fresh frozen sample. In some embodiments, the tumor sample DNA may be extracted from a bodily fluid. In some embodiments, the bodily fluid is blood, plasma, urine, cerebrospinal fluid, saliva, urine, or a combination thereof. In some embodiments, the tumor sample DNA may be extracted from a tissue. In some embodiments, the tissue is a tumor tissue.

In some embodiments, the DNA extracted from a bodily fluid may be cell-free DNA (cfDNA). In some embodiments, the DNA extracted from a bodily fluid may be genomic DNA (gDNA). In some embodiments, the tumor sample DNA may then be analyzed with a NGS technology workflow such as a whole genome sequencing (WGS), whole exome sequencing (WES), or targeted enrichment technology to sequence at least a specific subset of genomic regions associated with cancer diagnosis or prognosis. In embodiments including targeted enrichment, the tumor sample DNA may be assayed with a capture-based or an amplicon-based technology according to the assay provider protocols.

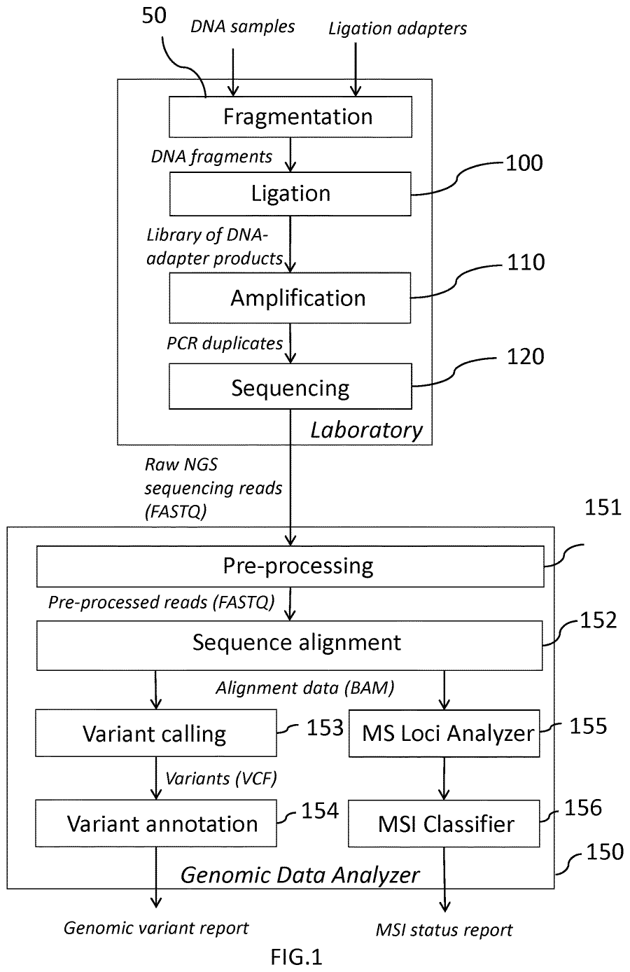

Such a genomic analysis workflow suitable for characterizing the microsatellite alteration status in genomic tumor samples possibly from low frequency DNA out of liquid biopsies is described with further detail with reference to FIG. 1. As will be apparent to those skilled in the art of DNA analysis, such a workflow comprises preliminary experimental steps to be conducted in a laboratory (also known as the “wet lab”) to produce DNA analysis data, such as raw sequencing reads in a next-generation sequencing workflow, as well as subsequent data processing steps to be conducted on the DNA analysis data to further identify information of interest to the end users, such as the detailed identification of DNA variants and related annotations, with a bioinformatics system (also known as the “dry lab”). Depending on the actual application, laboratory setup and bioinformatics platforms, various embodiments of a DNA analysis workflow are possible.

FIG. 1 describes an example of a workflow comprising a wet lab process wherein DNA samples are first fragmented with a fragmentation protocol 50 (optional) to produce DNA fragments.

Fragmentation 50 may result in a fragmented DNA being 50 to 1000 base-pairs in length. In some embodiments, fragmentation 50 may result in fragmented DNA being 50 base-pairs to 500 base-pairs in length. In some embodiments, fragmentation 50 may result in fragmented DNA being 500 to 1000 base-pairs in length.

Fragmentation 50 can be performed using any method known to the skilled artisan, including, but not limited to, mechanical shearing, sonication, ultrasonication, enzymatic fragmentation, partial digestion, restriction enzyme digestion. The fragmentation step 50 may be skipped if the starting material is already present as small fragments, as is for example typical of such as cDNA, cfDNA, and DNA isolated from some FFPE samples.

The DNA ends of these DNA fragments are then repaired and modified such as to be compatible with the adaptors that will be used. After fragmentation, the DNA fragments can be end-repaired or end-polished and a single adenine base can be added to form an overhang by an A-tailing reaction. This A-overhang allows adapters containing a single thymine base to pair with the DNA fragments.

Adaptors as will be further described in more detail throughout this enclosure may then be joined by ligation 100 to the DNA fragments in a reaction mixture, so as to produce a library of DNA-adaptor products, in accordance with some of the proposed methods.

In some embodiments, the adapters may include sample barcodes (also known as sample indexes) and/or exogenous molecular identifiers.

In some embodiments, the adaptors may include sample barcodes and/or exogenous molecular identifiers, with a number of distinct identifiers determined by the needs of the assay. In some embodiments, the sample barcodes and/or molecular identifiers may be 3 base pairs in length. In some embodiments, the molecular identifier tags may be 4 base pairs in length. In some embodiments, the sample barcodes and/or molecular identifiers may be 5 base pairs in length. In some embodiments, the sample barcodes and/or molecular identifiers be 6 base pairs in length. In some embodiments, the sample barcodes and/or molecular identifiers may be 7 base pairs in length. In some embodiments, the sample barcodes and/or molecular identifiers may be 8 base pairs in length. In some embodiments, the sample barcodes and/or molecular identifiers may be 9 base pairs in length. In some embodiments, the sample barcodes and/or molecular identifiers may be 10 base pairs in length. In some embodiments, the sample barcodes and/or molecular identifiers may be 11 base pairs in length. In some embodiments, the sample barcodes and/or molecular identifiers may be 12 base pairs in length. In some embodiments, the sample barcodes and/or molecular identifiers may be 13 base pairs in length. In some embodiments, the sample barcodes and/or molecular identifiers may be 14 base pairs in length. In some embodiments, the sample barcodes and/or molecular identifiers may be 15 base pairs in length. In some embodiments, the sample barcodes and/or molecular identifiers may be 16 base pairs in length. In some embodiments, the sample barcodes and/or molecular identifiers may be 17 base pairs in length. In some embodiments, the sample barcodes and/or molecular identifiers may be 18 base pairs in length. In some embodiments, the sample barcodes and/or molecular identifiers may be 19 base pairs in length. In some embodiments, the sample barcodes and/or molecular identifiers may be 20 base pairs in length.

In some embodiments, the sample barcodes and/or molecular identifiers and/or the adapters may comprise modified nucleic acids. Non-limiting examples of modified nucleic acids comprise locked nucleic acids (LNAs), which can fine-tune sequence melting temperatures, hybridization stability, resist degradation, etc., peptide nucleic acids (PNAs), which may enhance binding affinity, resist enzyme degradation, etc., 2′-O-methyloxy-ethyl bases (2′-MOE), which may offer increased binding affinity and resist nuclease degradation, fluorobases, which have a fluorine modified ribose for increased binding affinity, 5-hydroxybutynl-2′-deoxyuridine, which is a duplex-stabilizing modified base, and 8-aza-7-deazaguanosine, which is a modified base that eliminates secondary structures associated with GC-rich sequences.

The DNA library further undergoes amplification 110 and sequencing 120. The adaptor-ligated DNA fragments may then by amplified by PCR. The PCR primers might match the adapters on their 3′ ends, but be longer on their 5′ ends, for example to incorporate sample barcodes to the DNA fragments. After clean-up, the resulting collection of adaptor-ligated DNA fragments constitutes a WGS library. In some embodiments, the WGS library may be prepared using a PCR-free protocol.

Part, or the entirety, of the WGS library may be hybridized to capture probes to prepare a capture library. For example, preparation of the capture library may involve binding to streptavidin beads, extraction of DNA-bead complexes, and/or purification of the complexes. A PCR amplification may be conducted after the capture. Following removal of undesired reagents, the amplified DNA fragments constitute the capture library. It will be apparent to those skilled in the art that variation in the target-enrichment method may be implemented.

In some embodiments, part of the purified WGS library is mixed with the capture library, so that all genomic regions may be represented in the final library, although at a generally lower concentration for regions not targeted by the capture probes. This approach allows the combined generation of a low-pass WGS and target-enriched high-coverage sequence for selected regions.

The prepared library, which may contain some of the WGS library, in addition to the target-enrichment library, is subjected to high-throughput sequencing. To illustrate, the high-throughput sequencing may be accomplished via sequencing-by-synthesis, sequencing-by-ligation, or the like.

In some embodiments, part of the purified WGS library is sequenced in parallel to the capture library. In such embodiments, the WGS and capture libraries may be sequenced as part of distinct sequencing runs, or as part of the same sequencing runs after having received distinct sample barcodes. While this approach increases the handling time and sequence costs, it has the advantage of allowing distinguishing reads originating in the WGS library from those originating in the capture library after sequencing.

In a next generation sequencing workflow, the resulting DNA analysis data may be produced as a data file of raw sequencing reads in the FASTQ format. In some embodiments, the FASTQ files may be produced from BCL files. The workflow may then further comprise a dry lab Genomic Data Analyzer system 150, which takes as input the raw sequencing reads for a pool of DNA samples prepared with the ligation adaptors according to the proposed methods, and applies a series of data processing steps to identify genomic variants, for instance as a genomic variant report for the end user.

In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp). In some embodiments, the sequence reads are of a mean, median, or average length of about 1000 bp, 2000 bp, 5000 bp, 10,000 bp, or 50,000 bp or more.

Nanopore® sequencing methods and associated devices provided by Oxford Nanopore Technology PLC of Oxford, UK, for example, can provide sequence reads that can vary in size from tens to hundreds to thousands of base pairs. Illumina® parallel sequencing methods and associated devices provided by Illumina Inc. of San Diego, CA, for example, can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment. A sequencing read can correspond to a string of nucleotides at one or both ends of a nucleic acid fragment. A sequencing read can correspond to nucleotides of the entire nucleic acid fragment.

The Genomic Data Analyzer 150 uses at least one computer processor and may be implemented as a stand-alone computer software or multiple communicating computer software. An exemplary Genomic Data Analyzer system 150 is the SOPHIA Data Driven Medicine platform (SOPHIA DDM™) as already used by more than 1000 hospitals worldwide in 2019, but other systems may be used as well. Different detailed possible embodiments of data processing steps as may be applied by the Genomic Data Analyzer system 150 are described for instance in the international PCT patent application WO2017/220508, but other embodiments are also possible.

In a preferred embodiment, the Genomic Data Analyzer system 150 may first apply one or more pre-processing steps 151 to produce pre-processed reads from the raw sequencing reads inputs. The pre-processing steps may for instance comprise adaptor trimming, as well as read sorting, to analyze and group reads in families of reads representing potential PCR duplicates generated from the same original DNA fragments. In a possible embodiment, the raw reads as well as the pre-processed reads may be stored in the FASTQ file format, but other embodiments are also possible.

The Genomic Data Analyzer system 150 may further apply sequence alignment 152 to the pre-processed reads to produce read alignment data. In one embodiment, the read alignment data may be produced for instance in the BAM or SAM file format, but other embodiments are also possible. In some embodiments, sequence alignment might include local refinement of the alignment to align insertions/deletions among individual reads mapping to the same genomic window.

In some embodiments, sequencing reads representing potential PCR duplicates may be identified as part of the pre-processing steps 151 or after the sequence alignment 152. In some embodiments, identification of reads representing potential PCR duplicates may rely solely on endogenous molecular identifiers, such as the position of sequencing reads with respect to the reference genome and/or the sequence of the reads. As a non-limiting example, sequencing reads may be assigned to the same family of reads if they share the exact same start and end positions with respect to the reference genome. As another non-limiting example, sequencing reads may be assigned to the same family of reads if they share the exact same start and end positions with respect to the reference genome and their sequences are identical on a proportion of their length that exceeds a given threshold, such as 95% or 99%.

It will be apparent to those trained in the art that such read grouping can be performed in the presence or in the absence of exogenous molecular identifiers. In embodiments where exogenous molecular identifiers are incorporated during the library preparation, sequencing reads may be assigned to the same family of reads based on exogenous molecular identifiers or based on a combination of exogenous and endogenous molecular identifiers.

As a non-limiting example, sequencing reads may be assigned to the same family if they share the same exogenous barcodes and the same start and end positions with respect to the reference genome. As another non-limiting example, the exogenous molecular identifiers may differ by their length, and the length of the molecular identifiers inserted on both ends of the DNA fragments may be converted to numerical codes, following the method described in WO2021/053208.

The assignment of sequencing reads to families of reads may then be based solely on the numerical codes or on a combination of numerical codes and endogenous molecular identifiers, such as the start and end positions of reads with respect to the reference genome. It will be apparent to those skilled in the art that other existing methods previously described to identify potential PCR duplicates can be used.

The Genomic Data Analyzer system 150 may further apply variant calling 153 to the read alignment data to produce variant calling data. The read families assigned to sequenced reads may be used for either consensus sequencing or probabilistic sequencing, using methods known in the art. In one embodiment, the variant calling data may be produced for instance in the VCF file format, but other embodiments are also possible.

The Genomic Data Analyzer system 150 may further apply variant annotation 154 to the read alignment data to produce a genomic variant report for each DNA sample. In one embodiment, the genomic variant report may be visualized by the end user on a graphical user interface. In another possible embodiment, the genomic variant report may be produced as a text file for further data processing. Other embodiments are also possible.

In a preferred embodiment, the Genomic Data Analyzer system 150 may comprise an MS loci analyzer 155 to analyze the distributions of microsatellite biomarker loci lengths from the read alignment data. In some embodiments, the MS loci analyzer 155 may consider the read family information. The Genomic Data Analyzer system 150 may further comprise an MSI classifier 156 to produce an MSI status report for each DNA sample according to the classification of the analyzed microsatellite biomarker loci lengths distributions from the MS loci analyzer. In one embodiment, the MSI status report may be visualized by the end user on a graphical user interface. In another possible embodiment, the MSI status report may be produced as a text file for further data processing or communication. Other embodiments are also possible.

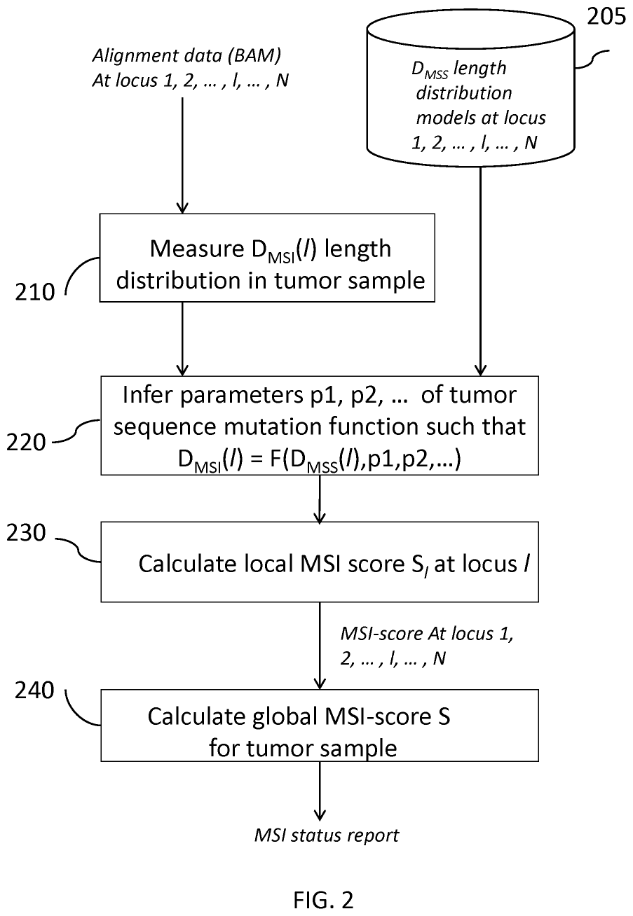

FIG. 2 illustrates a possible implementation of the bioinformatic workflow disclosed here to assess the MSI status of a sample. The repeat length distribution of the sample 210 is compared to the reference repeat length distribution 205 using a multiparameter model. At least two parameters are inferred from the model 220 and used to compute a MSI score for each locus 230. The locus-specific scores are aggregated to produce a global MSI score 240, which can be translated into a MSI status.

### Microsatellite Loci

The present disclosure provides for the detection of MSI out from a subset of microsatellite loci in the human genome. The microsatellite loci may be selected as homopolymers with a reference length in the human genome within a given range. As a non-limiting example, homopolymers with a reference length in the human genome between 10 bp and 100 bp may be selected. As another non-limiting example, homopolymers with a reference length between 10 and 50, 12 and 40, 14 and 30, or any other interval, may be selected. In a possible embodiment, the microsatellite loci may be homopolymers, short tandem repeats (STR) of heteropolymer, or a combination thereof.

In some embodiments, the microsatellite loci may be selected as homopolymers with a reference length in the human genome of at least 10 bp. In some embodiments, the microsatellite loci may be selected as homopolymers with a reference length in the human genome of at least 11 bp. In some embodiments, the microsatellite loci may be selected as homopolymers with a reference length in the human genome of at least 12 bp. In some embodiments, the microsatellite loci may be selected as homopolymers with a reference length in the human genome of at least 13 bp. In some embodiments, the microsatellite loci may be selected as homopolymers with a reference length in the human genome of at least 14 bp. In some embodiments, the microsatellite loci may be selected as homopolymers with a reference length in the human genome of at least 15 bp. In some embodiments, the microsatellite loci may be selected as homopolymers with a reference length in the human genome of at least 16 bp. In some embodiments, the microsatellite loci may be selected as homopolymers with a reference length in the human genome of at least 17 bp. In some embodiments, the microsatellite loci may be selected as homopolymers with a reference length in the human genome of at least 18 bp. In some embodiments, the microsatellite loci may be selected as homopolymers with a reference length in the human genome of at least 19 bp. In some embodiments, the microsatellite loci may be selected as homopolymers with a reference length in the human genome of at least 20 bp. In some embodiments, the microsatellite loci may be selected as homopolymers with a reference length in the human genome of at least 21 bp. In some embodiments, the microsatellite loci may be selected as homopolymers with a reference length in the human genome of at least 22 bp. In some embodiments, the microsatellite loci may be selected as homopolymers with a reference length in the human genome of at least 23 bp. In some embodiments, the microsatellite loci may be selected as homopolymers with a reference length in the human genome of at least 24 bp. In some embodiments, the microsatellite loci may be selected as homopolymers with a reference length in the human genome of at least 25 bp. In some embodiments, the minimal reference length for the homopolymers may exceed 25 bp.

In some embodiments, the microsatellite loci may be selected from STRs of a heteropolymer. In some embodiments, the heteropolymer is a dinucleotide repeat. In some embodiments, the heteropolymer is a trinucleotide repeat. In some embodiments, the heteropolymer is a tetranucleotide repeat. In some embodiments the heteropolymer is a pentanucleotide repeat. In some embodiments, the heteropolymer is a hexanucleotide repeat.

The microsatellite loci may be assayed from a body tissue sample, a liquid biopsy, a blood sample or a plasma sample. In the case of cancer diagnosis, the microsatellite loci may be assayed from cell-free DNA (cfDNA) or circulating tumor DNA (ctDNA) from a liquid biopsy, a blood sample or a plasma sample, or from a Formalin Fixed Paraffin Embedded (FFPE) or fresh frozen tumor tissue sample for each patient.

The microsatellite loci may be located in coding regions, non-coding regions, intronic regions, exons, or a combination thereof. The microsatellite loci may be analyzed from a tumor sample such as a with whole genome sequencing data (WGS), whole exome sequencing data (WES), amplicon-based targeted enrichment probes sequencing data, capture-based targeted enrichment sequencing data with various barcoding and molecular identification tagging technologies, such as single strand sequencing, double strand sequencing, duplex sequencing, circular sequencing, variable length tagging sequencing, and other methods known by those skilled in the art of NGS wet lab practice. Various high throughput sequencing technologies may be used, including those from Illumina, Ion Torrent Systems, Oxford Nanopore Technologies, Complete Genomics, Pacific Biosciences, and others, to produce a plurality of NGS reads of a predefined size (for instance, 50 pb, 100 bp, 150 bp, 200 bp, 250 bp, 300 bp and beyond) for each DNA fragment assayed from the input sample.

In some embodiments, the subset of microsatellite loci may be selected from the best performing microsatellite markers in MSI diagnosis for one or more cancers as reported in prior art work, for instance by Salipante in the mSINGS experiments or by Cortes-Ciriano. In some embodiments, the subset of microsatellite loci may be selected as the 10, 20, 30, 40, 50, 60, 70, 80, 90, 100, 110, 120, 130, 140, 150, 200, 250 best performing MSI homopolymer markers with a reference repetition length within the desired range.

In some embodiments, the subset of microsatellite loci selected for the computation of the MSI status may be pre-defined across assays. As a non-limiting example, capture probes or PCR primers designed specifically to cover these selected loci may be added to different target-enrichment assays. It will be apparent that the same loci can be assessed from whole-genome sequencing data without adding specific probes or primers.

In other embodiments, the subset of microsatellite loci selected for the computation of the MSI status may be defined independently for each wet lab protocol. As a non-limiting example, the best performing MSI markers among the set covered by the sequencing reads may be automatically selected based on a set of reference samples, including samples with known MSI statuses, processed with the wet lab protocol.

In yet other embodiments, the subset of microsatellite loci selected for the computation of the MSI status may be defined independently for each sample. As a non-limiting sample, the subset of MSI loci with the highest quality metrics may be automatically selected among those covered by the sequencing reads for the sample. Quality metrics may include coverage metrics, read-level quality scores, and the like. A combination of assay-level and sample-level metrics can be combined when selecting a subset of MSI loci for analyses.

### Estimation of Repeat Length Distributions

The repeat length distribution D_{MSI} can be computed, using a computer processor, for each of the subset of MSI loci selected for the computation of a MSI score.

The repeat length distribution D_{MSI} may be computed directly from the cleaned and trimmed reads aligned to the reference genome. As a non-limiting example, reads spanning the totality of the microsatellite (homo- or heteropolymer) may be identified as those including bases from both flanking regions. A minimum of 2 bp, 3 bp, 4 bp, 5 bp, or a different number of nucleotides matching each of the flanking regions may be required for a read to be considered as spanning the totality of the microsatellite.

In addition, or alternatively, the sum of the number of nucleotides matching each of the two flanking regions may be considered when assessing that a read spans the totality of the microsatellite. The number of nucleotides separating the two flanking regions may then be recorded as the repeat length of each read, and the repeat length distribution D_{MSI} may then be inferred as the distribution of repeat lengths among reads of the sample spanning the microsatellite.

In some embodiments, reads may be excluded from the distribution of repeat lengths if they do not meet certain quality criteria. For example, reads may be excluded from the calculations if their read-level quality score (e.g. phred score) is below a given threshold. As another example, reads may be excluded from the calculations if the identity of the nucleotides in the microsatellite of the read differs significantly from those constituting the microsatellite in the reference genome.

As a non-limiting example, if the microsatellite is a homopolymer in the reference genome, reads may be excluded from repeat length distribution calculations if their microsatellite includes at least one nucleotide that differs from the one constituting the reference homopolymer. Different quality criteria may be considered jointly to identify and exclude from the distribution of repeat lengths sequencing reads of low quality.

In embodiments where reads are assigned to families encompassing reads potentially representing PCR duplicates, the family information can be incorporated in the computation of the repeat length distribution D_{MSI}. In one embodiment, the length of the repeat may be calculated for each family. For each read spanning the microsatellite within a given family, the length of the repeat can be calculated as the number of nucleotides separating the two flanking regions.

As above, reads may be considered only if they cover both flanking regions on a number of bases that exceeds a pre-set threshold, and individual reads may be excluded based on other quality criteria. One repeat length value may then be inferred per family, for example by calculating the mean, the median, the mode, or another characteristic of the repeat lengths among retained reads of the family. In some embodiments, more than one value might be allowed by family, for example if multiple modes exist. The distribution of repeat length D_{MSI} may then be computed as the distribution of family-level values.

In some embodiments, incorporating family-level information, the value for a given family may only be included in the distribution of repeat length D_{MSI} if the family meets certain quality criteria. As a non-limiting example, the value of the family may be included only if the family encompasses more than a pre-set number of reads, and/or whether the family encompasses more than a pre-set number of reads originating from each of the two strands in the two original molecules (‘plus’ and ‘minus’ strands, also referred to as ‘Watson’ and ‘Crick’ strands).

As another non-limiting example, the value of the family may be included only if the spread of repeat lengths within the family is below a pre-set threshold. The spread may be estimated as the standard deviation, the variance, the percentage of reads differing from the mode (or other metric for the family), or with another method. As a further example, the quality criteria for including the value of the family may include a combination of the number of reads within the family, the spread of the repeat lengths within the family, the quality of individual reads (e.g. mean or median phred scores) and/or the identity of the nucleotides constituting the repeat in the reads as compared to the nucleotides constituting the repeat in the reference genome.

In some embodiments, the most likely repeat length of each family may be inferred with a model, such as a hidden Markov model or other Bayesian approaches, discriminative graphical models, deep learning and neural networks, state space models, or the like. With the family repeat lengths as the hidden states, such models may be parametrized to penalize the inference of numerous different repeat lengths among families.

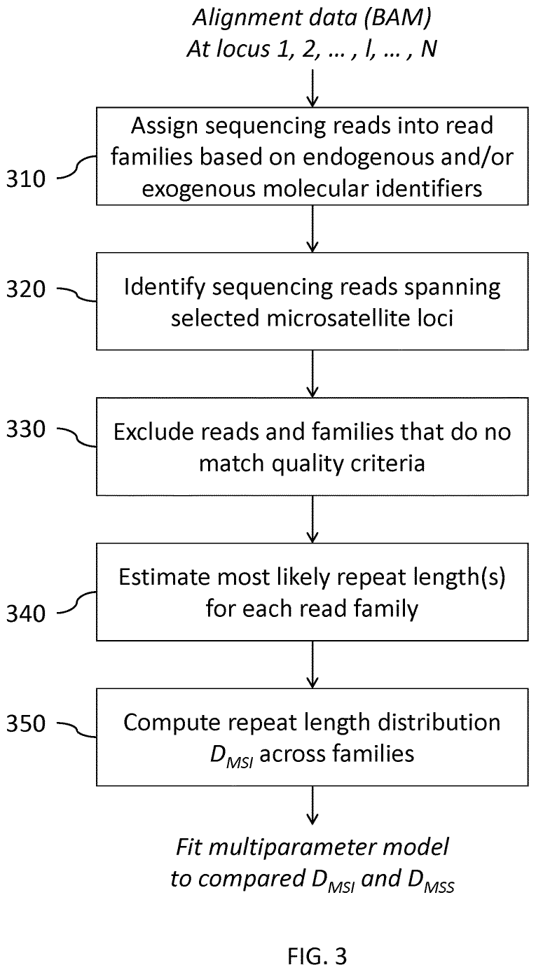

FIG. 3 illustrates a possible embodiment of a workflow to estimate the repeat length distribution D_{MSI} while accounting for the read family information. Sequencing reads are first assigned to read families based on endogenous and/or exogenous molecular identifiers 310. Sequencing reads spanning selected microsatellite loci are then identified 320. Reads within families and families do that not match selected quality criteria are then excluded 330. For each read family, the most likely repeat length is established 340. The repeat length distribution D_{MSI} is then computed from the family-level most likely repeat lengths 350.

In some embodiments, the read family information may be incorporated in the calculation of the repeat length distribution D_{MSI} by weighting read-level repeat lengths as a function of their representation within each family. As a non-limiting example, for each family, the percentage of reads matching the mode (or other family-level value) may be computed. The number of occurrences of each repeat length in the repeat length distribution D_{MSI} may then be computed as the sum of percentages of reads matching the mode across families having the repeat length as their mode.

As another non-limiting example, the percentage of reads within each family supporting each possible repeat length might be computed. The number of occurrences of each repeat length in the repeat length distribution D_{MSI} may then be computed as the sum of family-level percentages of reads supporting this length across families. Other mathematical operations may be used to integrate the family information in the calculation of the repeat length distribution D_{MSI}.

It will be apparent to those skilled in the art that the methods described here can be applied equally to the calculation of the sample repeat length distribution D_{MSI} and the reference repeat length distribution D_{MSS}. Depending on the type of sample available for the reference and the tumor, it may be preferrable to use different approaches for the calculation of D_{MSI} and D_{MSS}. As a non-limiting illustrative example, accounting for PCR duplicates may not be needed if the reference is obtained from germline blood samples, while PCR duplicates may need to be accounted for if the tumor sample is obtained from cfDNA.

In some embodiments, quality thresholds for inclusions of reads and/or families in the repeat length distributions may be optimized as part of the analyses. As a non-limiting example, some or all of the thresholds may be varied iteratively to identify the set of thresholds that minimize the difference between D_{MSS} and D_{MSI} while considering multiple microsatellite loci and/or multiple samples. As another non-limiting example, some or all of the thresholds may be varied iteratively to identify the set of thresholds that minimize the number of peaks in the repeat length distribution D_{MSI}. It will be apparent to those trained in the art that other optimization approaches may be used.

Models can be developed to compare the inferred D_{MSS} and D_{MSI} repeat length distributions. The method disclosed here relies on biologically relevant parametrization to accurately assess the MSI status across varying assays and in the presence of a mixture of germline and somatic mutations. The following sections describe the models, their parametrization, and their optimization, as will be implemented in a Genomic Data Analyzer using at least one computer processor.

### Background Model and Related Parameters

Due to the homopolymer or short tandem repeats, the NGS measurements tend to be noisy, so even in a healthy individual germline sample measurement, the measured homopolymer length at any microsatellite locus usually follows a variable length distribution instead of a constant number. Furthermore, this variable microsatellite length distribution may not exhibit a clear, unique peak of all read counts centered at the human genome reference microsatellite length.

The genomic data analyzer 150 may accordingly obtain a pre-calculated, background reference repeat length distribution, corresponding to the stable status in a given population: D_{MSS} for each microsatellite locus i. As will be apparent to those skilled in the art, this normal (MSS) repeat length distribution may be pre-computed offline from a set of normal samples in a given population, similar to some of the prior art peak finding methods, or across different populations so that it may be polymorphic, thus requiring an improved bioinformatics method more suitable than peak-finding methods to also characterize the MSI status at those more complex microsatellite loci.

If the locus is monomorphic in the reference population, the D_{MSS} distribution can be assumed to match a monomodal reference model. If the locus is polymorphic in the reference population, the D_{MSS} distribution may be assumed to match a multimodal model. There is therefore a different D_{MSS} stable reference length distribution at each microsatellite locus i, 1<=i<=N, where N is the total number of MSI loci to test.

In some embodiments, each D_{MSS} distribution histogram may be represented by a vector of read count values for each possible homopolymer length l at the microsatellite locus i, normalized relative to the total number of reads used in the length measurement at the microsatellite locus i. Other embodiments are also possible, for instance by measuring the relative abundance of individual length values at a locus relative to the read count of the most frequently occurring length value corresponding to the normalization reference value of 1, as proposed for instance in the MOSAIC method from Hause.

In somatic cells, the mutated DNA may include indels characterizing the MSI status at one or more microsatellite loci. For patient samples with an MSI inducing a somatic variant homopolymer length different from the MSS reference homopolymer length at a microsatellite locus i (for instance shortened or enlarged by 1 bp, 2 bp, 3 bp, etc), the genomic analysis may thus measure 210 a microsatellite length distribution D_{MSI}.

In some embodiments, each measured D_{MSI} histogram may be represented by a vector of read count values for each possible homopolymer length l at the microsatellite locus i, normalized relative to the total number of reads used in the length measurement at the microsatellite locus i. If the sample has no microsatellite instability at locus i, theoretically the measured somatic D_{MSI} is the same as the stable background reference model D_{MSS}. Conversely, if the sample has a microsatellite instability at locus i, the measured somatic D_{MSI} differs from the background reference model D_{MSS} (by a shift p of 1 bp, 2 bp, 3 bp, etc). It is therefore possible to estimate the patient sample MSI status at any given loci by comparing D_{MSI} to D_{MSS}, for instance by measuring a distance metrics between them.

As will be apparent to those skilled in the art, the prior art methods mentioned above achieve a direct statistical comparison between the D_{MSI} and D_{MSS}, using simple statistical measurements such as the mean, the variance or the standard deviation, without taking into account that multiple independent parameters may contribute to their difference. We propose to better characterize the underlying biological principles of an MSI event, by extracting MSI signatures from the genome that accounts for the different aspects that may impact the MSI status detection out from tumor samples. Preferably, those MSI signatures should reflect the underlying process of mutations happening in the genome, during the transformation from normal cells to somatic cells. The closer the genomic analysis model matches the biological events, the better the MSI characterization performance. To achieve this, for each sample, at each microsatellite locus i, the Genomic Data Analyzer 150 may infer a function F of at least two independent scalar parameters p₁, p₂ that can transform the background MSS distribution (D_{MSS}) into the measured MSI distribution (D_{MSI}) at that locus in the sample genomic data: F(D_{MSS}, p₁, p₂, . . . )=D_{MSI}.

To capture as closely as possible the underlying biological principles of MSI events, each parameter needs to be carefully designed. Moreover, the number of parameters needs to be carefully selected: with less parameters, it may not be possible to represent the biological principles behind the data, while more parameters may increase the risk of overfitting. In our experiments over two hundred microsatellite biomarker loci chosen from the literature (Salipante, Cortes-Ciriano), a model with 3 independent scalar parameters p₁, p₂, p₃ for the transform function provides a good enough fit. However, based on the data type, the definitions of the parameters and the number of parameters can be variable.

Prior art MSI detection approaches suffer from a number of limitations when applied to automatized genomic data analysis workflows from different laboratories possibly with different biological samples. Indeed, in practice, patient samples usually comprise a mixture of normal cells with germline DNA and somatic cells with mutated DNA, so the measured microsatellite length distribution D_{MSI} at any microsatellite locus i is partly due to the somatic DNA microsatellite homopolymer length contribution, partly due to the germline DNA microsatellite homopolymer length contribution.

The variant fraction corresponding to the ratio p₁ of somatic DNA in the patient sample may be variable per patient and may be relatively low in the case of cfDNA or ctDNA measurements. This complicates the evaluation of the actual length distribution characterizing the patient sample as the lower the somatic ratio p₁, the closer the measured D_{MSI} to the reference D_{MSS}. In other words, the distance between them is actually biased by the somatic variant fraction in the sample, in the case of a positive MSI status.

Next, a further parameter to take into account is how far the measured somatic D_{MSI} may differ from the background reference D_{MSS} (by a shift p₂ of 1 bp, 2 bp, 3 bp, etc) at a given microsatellite locus i where the patient sample may be characterized by a positive MSI status. A further variable p₂ may thus characterize the length distance between the main genomic alteration contributing to the measured length distribution D_{MSI} (corresponding to a biological MSI event in the somatic cell formation) and the reference D_{MSS} length lᵣ at locus i. Applying p₂ to the transform function will lead part of D_{MSS} (according to the somatic variant fraction ratio p₁) to move left or right by distance p₂ (direction defined by the sign of p₂), while the remaining part of D_{MSS} (according to the germline fraction ratio 1−p₁) stays at the same place. In another possible embodiment, p₂ may be the fraction of the length difference normalized by the reference homopolymer length lᵣ at locus i.

Moreover, even within the somatic cells for the same patient at a same microsatellite locus i, the deletion or the insertion of nucleotides in the homopolymer repeat sequences causing the MSI status are not always of the same length. For instance, in the exemplary case of an MSS reference peak length of 25 bp, the MSI status may be found with repeat lengths down to 15 bp, 16 bp, or 17 bp in different cells from the same patient sample.

This causes the measured length distribution D_{MSI} to exhibit one peak with lower height and larger width than assumed for instance in the prior art DPF-based method from Georgiadis. A further variable p₃ may thus characterize the variability in the length difference between different genomic alterations contributing to the measured length distribution D_{MSI}. By applying p₃ to the transform function, the p₁ part of the peak that moves out (by applying p₂) will exhibit a lower height and larger width.

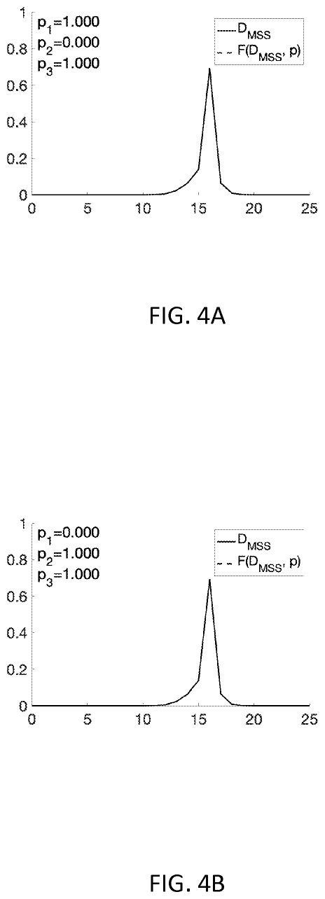

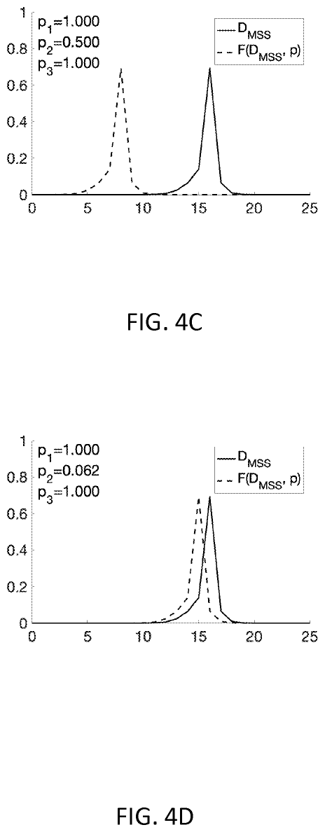

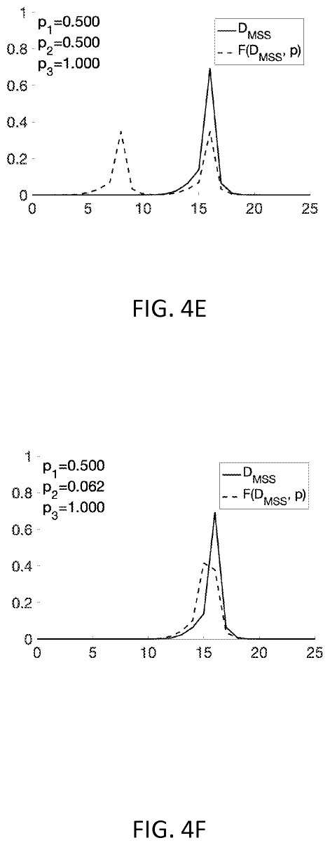

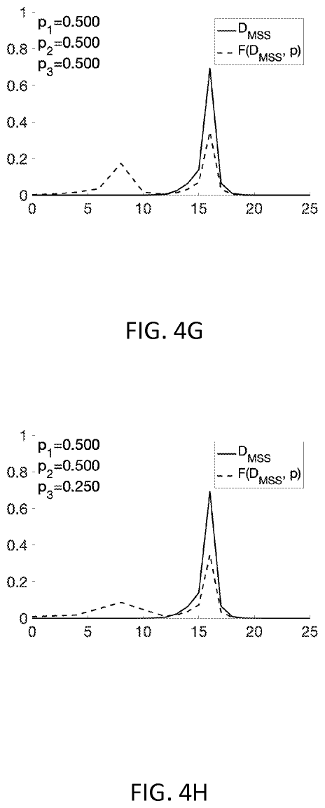

FIGS. 4A-4H illustrate the contributions of different p₁, p₂ and p₃ values to the transformed distribution F(D_{MSS}). In these simplified examples, germline samples follow the background reference length distribution D_{MSS}, p₁ is the ratio of somatic DNA in the sample DNA (variant fraction), p₂ is the length difference normalized by the reference homopolymer length at current locus (positive value means deletion and negative value means insertion) and p₃ means the stability of the length difference. By definition, applying p₁=0, p₂=₀ and p3=1 (p={0, 0, 1}) to F will generate exactly the same distribution as original. The locus in these examples has a germline homopolymer length of 16.

As illustrated by FIG. 4A (p={1, 0, 1}): if p2 equals to 0, even if p₁ is 100%, the transformed distribution will be the same as original. This is straightforward: if there is no mutation in somatic DNA (at least for this locus) there will be no difference no matter how variant fraction varies.

As illustrated by FIG. 4B (p={0, 1, 1}): if p₁ equals to 0, even if p₂ is 100%, the transformed distribution will be the same as original. This is also straightforward: if variant fraction is 0, the sample only has germline data so there will be no somatic difference measured no matter how much this variant will change in the somatic DNA.

As illustrated by FIG. 4C (p={1, 0.5, 1}): with a somatic variant fraction at 100% (p1=1) and a deletion of 8 bp (p2=0.5 on germline reference of 16 bp) the whole MSS distribution is shifted by 0.5*16=8 bps to the left.

As illustrated by FIG. 4D (p={1, 0.0625, 1}): with a somatic variant fraction at 100% (p1=1) and a deletion of 1 bp (p2=0.0625 on the germline reference of 16 bp) the whole MSS distribution is shifted 0.0625*16=1 bp to the left.

As illustrated by FIG. 4E (p={0.5, 0.5, 1}): with a somatic variant fraction at 50% (p1=0.5), half of the MSS distribution is shifted by 0.5*16=8 bps (p2=0.5 on the germline reference of 16 bp) to the left, and half of the MSS distribution remains unchanged. The transformed distribution exhibits accordingly 2 peaks, each peak having half of the original germline D_{MSS} peak height.

As illustrated by FIG. 4F (p={0.5, 0.0625, 1}): with a somatic variant fraction at 50%, (p1=0.5), half of the MSS distribution is shifted 0.0625*16=1 bp to the left (p₂=0.0625 on the germline reference of 16 bp), and half of the MSS distribution remains unchanged. The transformed distribution exhibits accordingly only 1 peak contributed from the two original peaks. However, as the 2 peaks are too close apart (only 1 bp difference) we cannot discriminate 2 separate peaks in the resulting transformed distribution.

Furthermore, in order to better fit real data without sharp peaks, a third parameter p₃ may be introduced to model the peak width, as there may be multiple somatic mutation events contributing to the somatic DNA such that the somatic data may exhibit a fuzzier peak than the reference background model data.

As illustrated by FIG. 4G (p={0.5, 0.5, 0.5}), with a somatic variant fraction at 50% (p1=0.5), half of the MSS distribution is shifted by 0.5*16=8 bps (p₂=0.5 on the background reference of 16 bp) to the left, and half of the MSS distribution remains unchanged (similar to FIG. 4E); the somatic peak attenuation parameter p₃=0.5 instead of 1 changes the shape of the somatic peak as half shorter but twice wider than the left somatic peak of FIG. 4E, while the background reference peak (right small peak in FIG. 4E) will remain unchanged.

Similarly, as illustrated by FIG. 4H (p={0.5, 0.5, 0.25}), the somatic peak attenuation parameter p₃=0.25 instead of 1 changes the shape of the somatic peak as one quarter shorter but four times wider, while the germline peak remains unchanged.

As will be apparent to those skilled in the art, the values of p₁, p₂, p₃ as described in the examples of FIGS. 4A-4H may be interchangeable. They may also be measured as absolute values or relative to a reference. In an example (not illustrated) of using only two parameters p₁, p₂ as the variant fraction and the MSI shifted length relative to the reference MSS homopolymer length lᵣ at locus i, for each possible homopolymer length l (0≤l≤lₘ) F may be defined as:

$$F ( {D}_{MSS} , {p}_{1} , {p}_{2} ) ( l ) = ( 1 - {p}_{1} ) * {D}_{MSS} ( l ) + p 1 * {D}_{MSS} ( l + {p}_{2} * {l}_{r} ) if 0 \leq l + {p}_{2} * {l}_{r} \leq {l}_{m}$$

$$F ( {D}_{MSS} , {p}_{1} , {p}_{2} ) ( l ) = ( 1 - {p}_{1} ) * {D}_{MSS} ( l ) if l + {p}_{2} * {l}_{r} \leq 0 or if l + {p}_{2} * {l}_{r} \geq {l}_{m}$$

where lᵣ is the reference background model of the MSS (stable status) homopolymer length at this locus and lₘ is the maximum homopolymer length at this locus.

In the example of FIG. 4, for each homopolymer length l (0≤l≤lₘ) at one locus i, F may be defined as:

$$F ( {D}_{MSS} , {p}_{1} , {p}_{2} , {p}_{3} ) ( l ) = ( 1 - {p}_{1} ) * {D}_{MSS} ( l ) + {p}_{1} * {p}_{3} * {D}_{MSS} ( {l}_{r} + {p}_{2} * {l}_{r} + {p}_{3} * ( l - {l}_{r} ) ) if 0 \leq {l}_{r} + {p}_{2} * {l}_{r} + {p}_{3} * ( l - {l}_{r} ) \leq {l}_{m} ,$$

$$and F ( {D}_{MSS} , {p}_{1} , {p}_{2} , {p}_{3} ) ( l ) = ( 1 - {p}_{1} ) * {D}_{MSS} ( l ) else ,$$

where lᵣ is the reference (MSS stable) homopolymer length at this locus and lₘ is the maximum homopolymer length at this locus.

### Inferring the Model Parameters to Characterize MSI Events

After the parameters in the transform function model have been clearly defined, we need to measure the values of these parameters that can minimize the difference between the measured sample distribution D_{MSI} and the background germline theoretical distribution D_{MSS} by applying the transform function F to the background reference length distribution model D_{MSS} at each locus i 205.

In some embodiments, at each microsatellite locus, the MS Loci Analyzer 155 may apply a curve-fitting method by minimizing a distance metric between the transformed D_{MSS} reference distribution and the measured D_{MSI} length distribution, out of possible models calculated with F according to different sets of parameters p={p₁, p₂, . . . } to derive actual values for the parameters p. Various curve fitting methods may be used, for Ln-norm fitting, where n can be different numbers; n=2 for non-linear least-squares curve-fitting, including ordinary least squares (wherein all observations are equally treated) or weighted least squares methods (wherein observations are not equally treated but rather weighted); n=1 for minimization of the sum of absolute differences (SAD; or least absolute residuals).

In some embodiments, assuming that for each homopolymer locus we may have a different biological parametrization p={p₁, p₂, p₃}, we can infer p by non-linear least-squares curve-fitting:

$${min}_{p} \{ {\sum }_{i = 0}^{{l}_{m}} {( F ( {D}_{MSS} , p ) ( l ) - {D}_{MSI} ( l ) )}^{2} \}$$

where lₘ is the maximum homopolymer length at this locus.

In another possible embodiment, for example, if there are only 2 parameters instead of 3, or if the required precision is not very high, a brute force search algorithm may alternately be applied to derive 220 the parameters instead of using a curve-fitting method.

### MSI Characterization

As mentioned above, the distribution transformation algorithm can better describe the biological principle of MSI events. In the above example, p₁ is defined as the ratio of somatic DNA in the patient sample, thus can be considered as how much the MSI event has spread among this patient; p₂ is defined as the distance between the homopolymer length of MSI status and MSS status, thus can be considered as how severe is the MSI status (in early stage or in late stage); p₃ is not directly correlated with the MSI status, but, in the curve-fitting step, introducing this additional parameter p₃ helps to determine p₁ and p₂ more accurately out of real data.

In some embodiments, the product of p₁ and p₂ may be used to characterize the MSI event. For each locus i, a local MSI score sⁱ may then be calculated 230 as:

$${s}^{i} = {p}^{i} \cdot {p}^{i}$$

In some embodiments, for each sample, the MSI Classifier 156 may calculate 240 a raw global MSI score S over all microsatellite loci, to characterize the MSI status:

$$S = {\sum }_{i = 1}^{N} {s}^{i} = {\sum }_{i = 1}^{N} {p}_{1}^{i} \cdot {p}_{2}^{i}$$

where N is the total number of selected loci. In another possible embodiment, S may be normalized by the average MSI score of background reference (MSS) samples. In another possible embodiment, S may be calculated as the mean or median of sⁱ across the analyzed loci. Other normalization methods are also possible.

In some embodiments, the MSI classifier may also calculate 240 the global score S by counting the number of loci with positive MSI signal, defined by a predefined threshold value on the local MSI status sⁱ score for each locus i. The predefined threshold at each locus may be predetermined based on parameters inputted for the Genomic Data Analyzer 150. As a non-limiting example, the threshold may vary for different cancer types. Other embodiments are also possible.

In general, the higher the score S for a patient sample, the higher the likelihood that MSI events have happened in this sample. The MSI classifier 156 may classify MSI events according to a cut-off value, and report the MSI status as positive according to how the score compares to the cut-off value. The Genomic Data Analyzer 150 may also report the MSI status as positive (MSI-High) if the global score exceeds a first predefined cutoff value, as negative (MSS) if the global score is below a second predefined cutoff value, or in-between (MSI-Low) if the global score is between the first and the second cutoff values. The predefined cutoff values for the global score may be predetermined parameters for the Genomic Data Analyzer 150, e.g. pre-calculated for different cancer types. Other embodiments are also possible.

### Implementation of the Method in a Computer-Based Genomic Data Analyzer

At least one computer processor is required to perform the dry lab steps of the instant method. In some embodiments, a stand-alone computer software may be used to compute MSI scores from cleaned sequencing reads aligned to a reference genome. In other embodiments, the computer software, representing the Genomic Data Analyzer 150, may also perform the pre-processing and sequence alignment steps. In some embodiments, the Genomic Data Analyzer may be integrated in a genomic analysis platform, where other components may be responsible for performing different types of analyses, such as the detection of short variants (e.g. SNPs and short indels), the detection of copy number variants, the detection gene fusion and structural rearrangements, the estimation of a tumor mutational burden, the assessment of genomic instability, and/or the annotation of detected variants to return, among other, their predicted pathogenicity. The Genomic Data Analyzer may moreover by connected to a user interface, enabling the user to explore the results, produce different graphs, produce automated or customized reports, and export, download, or print the data, results, and reports.

In some embodiments, the steps of the Genomic Data Analyzer 150 may be carried out by a software device located on a cloud computing server to permit decentralized analysis. In an embodiment, the cloud computing server may comprise a global center that provides central services, such as user authentication and authorization. In one embodiment, the cloud computing server may comprise at least one regional center to provide file management, storage, and other functionalities. It is contemplated that this permits the users to access the software from a server that complies with local requirements and regulations.

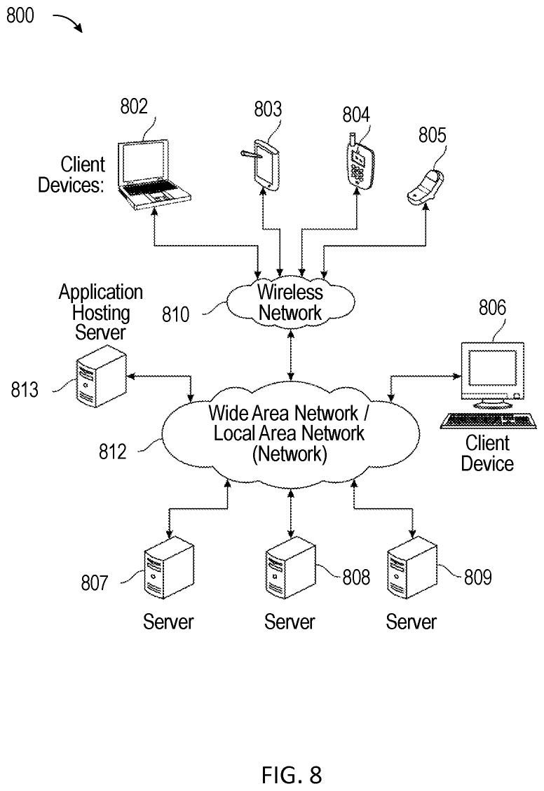

FIG. 8 illustrates components of one embodiment of an environment in which the dry lab steps of the present disclosure may be practiced. Not all of the components may be required to practice the present disclosure, and variations in the arrangement and type of the components may be made without departing from the spirit or scope of the present disclosure. As shown, the system 800 includes one or more Local Area Networks (“LANs”)/Wide Area Networks (“WANs”) 812, one or more wireless networks 810, one or more wired or wireless client devices 806, mobile or other wireless client devices 802-805, servers 807-809, and may include or communicate with one or more data stores or databases. The client devices 802-806 may include, for example, at least one of desktop computers, laptop computers, set top boxes, tablets, cell phones, smart phones, smart speakers, wearable devices (such as the Apple Watch) and the like. Servers 807-809 can include, for example, one or more application servers, content servers, search servers, and the like. FIG. 8 also illustrates application hosting server 813.

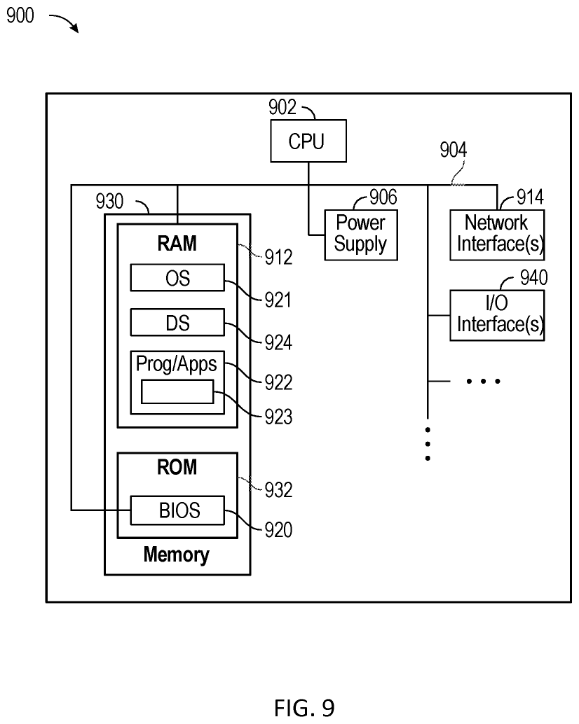

FIG. 9 illustrates a block diagram of an electronic device 900 that can implement one or more aspects of an apparatus, system, and method for measurement and secure transmission of physical properties (the “Engine”) according to one embodiment of the present disclosure. Instances of the electronic device 900 may include servers, e.g., servers 807-809, and client devices, e.g., client devices 802-806. In general, the electronic device 900 can include a processor/CPU 902, memory 930, a power supply 906, and input/output (I/O) components/devices 940, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 900. A touchscreen may determine whether a user is providing input by, for example, determining whether the user is touching the touchscreen with a part of the user's body such as his or her fingers. The electronic device 900 can also include a communications bus 904 that connects the aforementioned elements of the electronic device 900. Network interfaces 914 can include a receiver and a transmitter (or transceiver), and one or more antennas for wireless communications.

The processor 902 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 930, which can include Random Access Memory (RAM) 912 and Read Only Memory (ROM) 932, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 921, data storage 924, which may include one or more databases, and programs and/or applications 922, which can include, for example, software aspects of the program 923. The ROM 932 can also include Basic Input/Output System (BIOS) 920 of the electronic device.

Software aspects of the program 923 are intended to broadly include or represent all programming, applications, algorithms, models, software, and other tools necessary to implement or facilitate methods and systems according to embodiments of the present disclosure. The elements may exist on a single computer or be distributed among multiple computers, servers, devices, or entities.

The power supply 906 contains one or more power components and facilitates supply and management of power to the electronic device 900.

The input/output components, including Input/Output (I/O) interfaces 940, can include, for example, any interfaces for facilitating communication between any components of the electronic device 900, components of external devices (e.g., components of other devices of the network or system 800), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 940 and the bus 904 can facilitate communication between components of the electronic device 900, and in an example can ease processing performed by the processor 902.

Where the electronic device 900 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system, and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 802-806, as may be used in an example apparatus, system and method embodying the Engine, may range widely in terms of capabilities and features. For example, a cell phone, smart phone, or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 802-806, for example, as may be used in an example apparatus, system and method implementing the Engine, may run a variety of operating systems, including personal computer operating systems such as Windows, iOS or Linux, and mobile operating systems such as iOS, Android, Windows Mobile, and the like.

Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games (such as fantasy sports leagues), receiving advertising, watching locally stored or streamed video, or participating in social networks.

In example aspects of the apparatus, system and method implementing the Engine, one or more networks, such as networks 810 or 812, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 810, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present disclosure include apparatuses, systems, and methods implementing the Engine. Embodiments of the present disclosure may be implemented on one or more of client devices 802-806, which are communicatively coupled to servers including servers 807-809. Moreover, client devices 802-806 may be communicatively (wirelessly or wired) coupled to one another. In particular, software aspects of the Engine may be implemented in the program 923. The program 923 may be implemented on one or more client devices 802-806, one or more servers 807-809, and 813, or a combination of one or more client devices 802-806, and one or more servers 807-809 and 813.

In an embodiment, the system may receive, process, generate and/or store time series data. The system may include an application programming interface (API). The API may include an API subsystem. The API subsystem may allow a data source to access data. The API subsystem may allow a third-party data source to send the data. In one example, the third-party data source may send JavaScript Object Notation (“JSON”)-encoded object data. In an embodiment, the object data may be encoded as XML-encoded object data, query parameter encoded object data, or byte-encoded object data.

### Practical Applications

In some embodiments, the methods and systems described herein are used to determine an MSI score of a subject. In some embodiments, the subject is suffering from, or suspected to suffer from, cancer.

In some embodiments, cancer types can be grouped into broader categories, e.g., carcinoma (meaning a cancer that begins in the skin or in tissues that line or cover internal organs, and its subtypes, including adenocarcinoma, basal cell carcinoma, squamous cell carcinoma, and transitional cell carcinoma); sarcoma (meaning a cancer that begins in bone, cartilage, fat, muscle, blood vessels, or other connective or supportive tissue); leukemia (meaning a cancer that starts in blood-forming tissue (e.g., bone marrow) and causes large numbers of abnormal blood cells to be produced and enter the blood); lymphoma and myeloma (meaning cancers that begin in the cells of the immune system); and central nervous system cancers (meaning cancers that begin in the tissues of the brain and spinal cord).

Examples of carcinomas include, without limitation, giant and spindle cell carcinoma, small cell carcinoma, papillary carcinoma, squamous cell carcinoma, lymphoepithelial carcinoma, basal cell carcinoma, pilomatrix carcinoma, transitional cell carcinoma, papillary transitional cell carcinoma, an adenocarcinoma, a gastrinoma, a cholangiocarcinoma, a hepatocellular carcinoma, a combined hepatocellular carcinoma and cholangiocarcinoma, a trabecular adenocarcinoma, an adenoid cystic carcinoma, an adenocarcinoma in adenomatous polyp, an adenocarcinoma, familial polyposis coli, a solid carcinoma, a carcinoid tumor, a branchiolo-alveolar adenocarcinoma, a papillary adenocarcinoma, a chromophobe carcinoma, an acidophil carcinoma, an oxyphilic adenocarcinoma, a basophil carcinoma, a clear cell adenocarcinoma, a granular cell carcinoma, a follicular adenocarcinoma, a non-encapsulating sclerosing carcinoma, adrenal cortical carcinoma, an endometroid carcinoma, a skin appendage carcinoma, an apocrine adenocarcinoma, a sebaceous adenocarcinoma, a ceruminous adenocarcinoma, a mucoepidermoid carcinoma, a cystadenocarcinoma, a papillary cystadenocarcinoma, a papillary serous cystadenocarcinoma, a mucinous cystadenocarcinoma, a mucinous adenocarcinoma, a signet ring cell carcinoma, an infiltrating duct carcinoma, a medullary carcinoma, a lobular carcinoma, an inflammatory carcinoma, Paget's disease, a mammary acinar cell carcinoma, an adenosquamous carcinoma, an adenocarcinoma w/squamous metaplasia, a sertoli cell carcinoma, embryonal carcinoma, choriocarcinoma.

Examples of sarcomas include, without limitation, glomangiosarcoma, sarcoma, fibrosarcoma, myxosarcoma, liposarcoma, leiomyosarcoma, rhabdomyosarcoma, embryonal rhabdomyosarcoma, alveolar rhabdomyosarcoma, stromal sarcoma, carcinosarcoma, synovial sarcoma, hemangiosarcoma, kaposi's sarcoma, lymphangiosarcoma, osteosarcoma, juxtacortical osteosarcoma, chondrosarcoma, mesenchymal chondrosarcoma, giant cell tumor of bone, ewing's sarcoma, odontogenic tumor, malignant, ameloblastic odontosarcoma, ameloblastoma, malignant, ameloblastic fibrosarcoma, myeloid sarcoma, mast cell sarcoma.

Examples of leukemias include, without limitation, leukemia, lymphoid leukemia, plasma cell leukemia, erythroleukemia, lymphosarcoma cell leukemia, myeloid leukemia, acute myeloid leukemia, basophilic leukemia, eosinophilic leukemia, monocytic leukemia, mast cell leukemia, megakaryoblastic leukemia, myelodysplastic syndrome, and hairy cell leukemia.

Examples of lymphomas and myelomas include, without limitation, malignant lymphoma, Hodgkin's disease, Hodgkin's, paragranuloma, malignant lymphoma, small lymphocytic, malignant lymphoma, large cell, diffuse, malignant lymphoma, follicular, mycosis fungoides, other specified non-Hodgkin lymphomas, myeloma, dark zone lymphoma, and multiple myeloma.

Examples of melanomas include, without limitation, malignant melanoma, amelanotic melanoma, superficial spreading melanoma, malignant melanoma in giant pigmented nevus, and epithelioid cell melanoma.

Examples of brain/spinal cord cancers include, without limitation, pinealoma, malignant, chordoma, glioma, malignant, ependymoma, astrocytoma, protoplasmic astrocytoma, fibrillary astrocytoma, astroblastoma, glioblastoma, oligodendroglioma, oligodendroblastoma, primitive neuroectodermal, cerebellar sarcoma, ganglioneuroblastoma, neuroblastoma, retinoblastoma, olfactory neurogenic tumor, meningioma, malignant, neurofibrosarcoma, neurilemmoma, malignant.

Examples of other cancers include, without limitation, a thymoma, an ovarian stromal tumor, a the coma, a granulosa cell tumor, an androblastoma, a leydig cell tumor, a lipid cell tumor, a paraganglioma, an extra-mammary paraganglioma, a pheochromocytoma, blue nevus, malignant, fibrous histiocytoma, malignant, mixed tumor, malignant, mullerian mixed tumor, nephroblastoma, hepatoblastoma, mesenchymoma, malignant, brenner tumor, malignant, phyllodes tumor, malignant, mesothelioma, malignant, dysgerminoma, teratoma, malignant, struma ovarii, malignant, mesonephroma, malignant, hemangioendothelioma, malignant, hemangiopericytoma, malignant, chondroblastoma, malignant, granular cell tumor, malignant, malignant histiocytosis, immunoproliferative small intestinal disease, and myeloproliferative neoplasm.

In some embodiments, the method further comprises selecting based on the computed MSI score, one or more treatment options for a subject in need of a cancer treatment. In some embodiments, the selection of treatment options may be based on the computed MSI score together with any combinations or other genomic variants, any combination of genetic variants, histological data, pathological data, radiological data, clinical data, metabolic data, or other data for the subject.

In some embodiments, the method further comprises administering to a subject in need thereof, a cancer treatment for a hematologic malignancy or a solid tumor malignancy. In some embodiments, the cancer treatment is administered to a subject in need thereof for treating a tumor, e.g., a malignant or non-malignant tumor.

In some embodiments, the cancer treatment comprises a chemotherapy, a radiation therapy, an immunotherapy, a hormonal therapy, a targeted therapy, a cell therapy, or a stem cell transplant.

Nonlimiting examples of chemotherapies include alkylating agents (e.g., altretamine, bendamustine, busulfan, carboplatin, chlorambucil, cisplatin, cyclophosphamide, dacarbazine, ifosfamide, mechlorethamine, melphalan, oxaliplatin, procarbazine, temozolomide, thiotepa, trabectedin, carmustine, lomustine, streptozocin), antimetabolites (e.g., 5-fluorouracil, 6-mercaptopurine, azacitidine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, trifluridine), topoisomerase inhibitors (e.g., etoposide, irinotecan, irinotecan liposomal, mitoxantrone, teniposide, topotecan), mitotic inhibitors (e.g., cabazitaxel, docetaxel, nab-paclitaxel, paclitaxel, vinblastine, vincristine, vincristine liposomal, vinorelbine), antitumor antibiotics (e.g., daunorubicin, doxorubicin, doxorubicin liposomal, epirubicin, idarubicin, mitoxantrone, valrubicin, bleomycin, dactinomycin, mitomycin-c), and other chemotherapies (e.g., all-trans-retinoic acid, arsenic trioxide, asparaginase, eribulin, ixabepilone, mitotane, omacetaxine, pegaspargase, procarbazine, romidepsin, vorinostat).

Nonlimiting examples of radiation therapy include external beam radiation therapy (e.g., CD conformal radiation therapy, intensity-modulated radiation therapy, image-guided radiation therapy, stereotactic radiotherapy, proton therapy, stereotactic body radiation therapy, volumetric modulated arc therapy, tomotherapy), superficial radiotherapy, intraoperative radiotherapy, and internal radiation therapy (e.g., brachytherapy, interstitial brachytherapy, intracavitary brachytherapy, intraluminal brachytherapy, systemic radiation therapy, radioimmunotherapy, radiopharmaceutical therapies).

Nonlimiting examples of immunotherapy include checkpoint inhibitors (e.g., nivolumab, pembrolizumab, ipilimumab), adoptive cell therapy (e.g., CAR T cell therapy, TIL therapy), monoclonal antibodies (e.g., rituximab, trastuzumab, cetuximab), vaccines (e.g., HPV vaccine, melanoma vaccine), cytokines (e.g., interferon, interleukin), oncolytic viruses (e.g., talimogene la-KS), and immune modulators (e.g., lenalidomide).

Nonlimiting examples of hormonal therapy include aromatase inhibitors (e.g., anastrozole, exemestane, letrozole), selective estrogen receptor modulators (e.g., tamoxifen), luteinizing hormone-releasing hormone agonists (e.g., goserelin, leuprolide), anti-androgens (e.g., bicalutamide, flutamide, nilutamide), progestins (e.g., medroxyprogesterone, megestrol), androgen synthesis inhibitors (e.g., abiraterone, ketoconazole), and CDK 4/6/inhibitors (e.g., ribociclib, abemaciclib).

Nonlimiting examples of targeted therapy include monoclonal antibodies (e.g., trastuzumab, cetuximab, bevacizumab, pembrolizumab), small molecule drugs (e.g., tyrosine kinase inhibitors, proteasome inhibitors, PARP inhibitors, menin inhibitors, IDH1/IDH2 inhibitors), antibody-drug conjugates (e.g., trastuzumab emtansine), and angiogenesis inhibitors.

As will be apparent to those skilled in the art of bioinformatics, while the proposed methods and examples have been described primarily for microsatellite loci comprising homopolymer (mononucleotide) repeats, they may also be applied to microsatellite loci comprising heteropolymer or short tandem repeats, the repeat length distribution referring to the number of repeats of the heteropolymer sequences rather than the number of repeats of the homopolymer nucleotide.

As will be apparent to those skilled in the art of genomics, while the disclosed method does not require paired tumor-normal samples, the method is compatible with paired tumor-normal samples. While the envisioned reference repeat length distribution D_{MSS} is based on a reference population that differs from the tumor subject, D_{MSS} may also be inferred from a paired normal sample of the same subject. In a possible embodiment, the reference repeat length distribution D_{MSS} may be based on multiple non-tumor samples of the subject. In another possible embodiment, the reference repeat length distribution D_{MSS} may be based on a baseline sample in a serial biopsy dataset. It will be apparent that the model can easily be configured to detect decreases of the MSI score, for example following a treatment. The instant method may therefore support monitoring of the MSI status through longitudinal series.

As will be apparent to those skilled in the art of bioinformatics, a diversity of genomic data analyzer workflows may employ the proposed methods, possibly in combination with probabilistic sequencing and/or probabilistic variant calling methods. In a possible embodiment, a probabilistic classifier may be trained to calculate the global MSI score and/or to report the MSI status according to the local MSI scores across the N loci. In some possible embodiments, the probabilistic classifiers may take into account the read families based on unique molecular identifiers.

While the instant invention discloses the transformation of a quantitative MSI score into a qualitative MSI status, the quantitative MSI score obtained with the present method may be combined with other evidence when assessing the MSI status, or when predicting prognosis, treatment effects, and the like. In a possible embodiment, the MSI status may be computed through a combination of the MSI score computed using the method disclosed here and other evidence obtained from variant analyses of genes in the mismatch repair system, tumor mutational burden estimations, global or marker-specific hypo- or hyper-methylation, digital analysis of stained slides, and the like. In a possible embodiment, the MSI score computed using the method disclosed here may be integrated into multimodal machine learning models trained to predict the prognosis, clinical events, or treatment responses of subjects based on combinations of different genomic markers, in some case alongside imaging, clinical, pathological, proteomics, methylomics, and/or transcriptomics data.

### EXAMPLES

The following examples are put forth to provide those of ordinary skill in the art with a complete disclosure and description of how to make and use the invention of the present disclosure and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

The example provided below is a nonlimiting example intended to illustrate one possible embodiment of the method or system described herein. It should not be construed as restricting the scope of said method or system, which are not limited to the specific details or configurations presented below. The method or system described herein encompass any modifications, variations, or alternatives that would be apparent to those skilled in the art, while maintaining the spirit and intended purposes of said method or system.

### Example 1

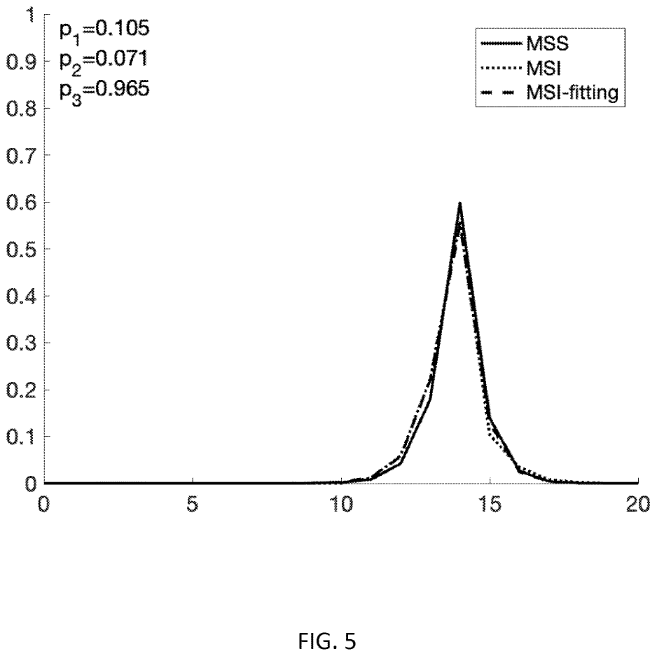

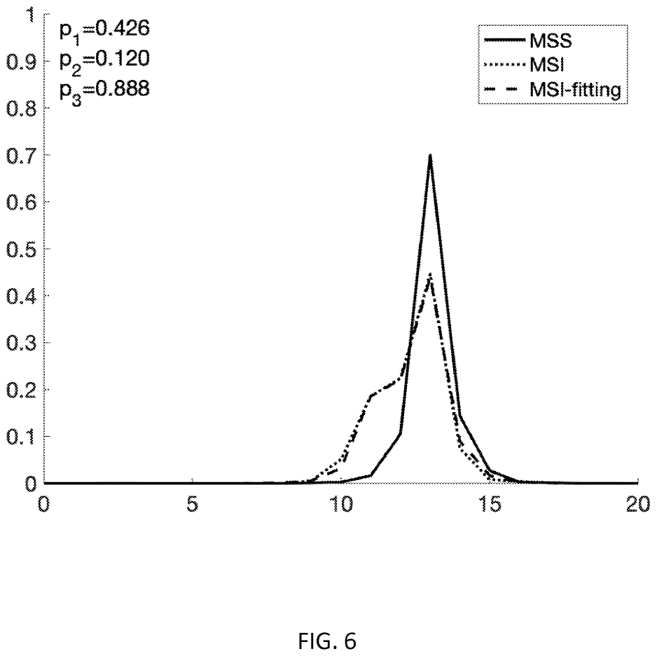

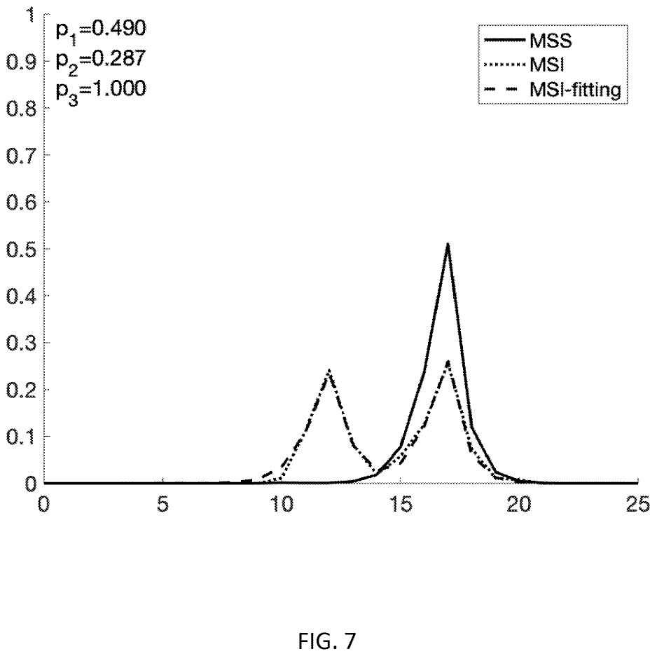

FIG. 5, FIG. 6, and FIG. 7 illustrate the D_{MSS} distribution, the D_{MSI} distribution from the patient sample, and the MSI-fitting distribution F(D_{MSS}, p₁, p₂, p₃) at 3 different microsatellite loci. In these figures, p₁ represents the ratio of somatic DNA (variant fraction), p₂ represents the length difference normalized by the reference homopolymer length at current locus (positive value means deletion and negative value means insertion), and p₃ represents the stability of the length difference.

In the example of FIG. 5, the proposed method infers parameters p₁=0.105 for the somatic variant fraction, p₂=0.071 measuring a deletion of 7.1% of 14 bp=0.99 bp in average and p₃=0.965 for a more precise p₁ and p₂ fitting. The MSI score at this locus, calculated as the product of p₁ and p₂, is then p₁*p₂=0.00745.

Similarly, in FIG. 6, the proposed method infers parameters p₁=0.426 for the somatic variant fraction, p₂=0.120 measuring a deletion of 12.0% of 13 bp=1.6 bp in average and p₃=0.888 for a more precise p₁ and p₂ fitting. The MSI score at this locus, calculated as the product of p₁ and p₂, is then p₁*p₂=0.05112, thus significantly higher than the one of FIG. 5.

Moreover, in the example of FIG. 7, the proposed method infers parameters p₁=0.490 for the somatic variant fraction, p₂=0.287 measuring a deletion of 28.7% of 17 bp=4.9 bp in average and p₃=1.000 for a more precise p₁ and p₂ fitting. The MSI score at this locus, calculated as the product of p₁ and p₂, is then p₁*p₂=0.140, thus significantly higher than the ones of FIG. 5 or FIG. 6. It is, therefore possible to quantify the MSI status accurately at each locus, independently of the magnitude of the homopolymer length variation.

shows experimental results of the proposed MSI status analysis method benchmarked against the Promega test for a number of different tumor FFPE samples of different cancer origins (endometrial, ovarian, colon, uterus). The samples have been assayed with the capture-based kit from SOPHIA GENETICS on a subset of microsatellite loci identified in the Salipante and the Cortes-Ciriano prior art, and sequenced with the Illumina MiSeq sequencer to provide 150 bp long reads at a coverage of around 3000× at each locus. After read pre-processing and alignment, the read length distribution at each of the selected loci has been measured and fitted with LSE onto the background MSS stable length distribution model at the corresponding locus to derive three best matching parameters p₁, p₂ and p₃ (the same definition as described in FIG. 4, FIG. 5, FIG. 6, and FIG. 7) and a MSI score S over all the selected loci has been derived for each sample according to the proposed methods.

The MSI Classifier has been configured to report a positive MSI-status above a cut-off value of 0.5 on the normalized MSI score value S, and to report a negative MSI-status below that value. Alternately, a cut-off value of 0.07 on the raw MSI score value S may be used by the Classifier. Other values are also possible, depending on the application and whether normalization has been applied.

As reported in, the proposed MSI Classifier gives 100% sensitivity and 100% specificity over the tested pool of samples.

TABLE 1
Comparison of the MSI status estimation with the proposed
NGS-based MSI-Score Classifier Comparison and the PROMEGA
reference test over a pool of different tumor samples.

| | | MSI-Score | MSI-Score | MSI-Score | |
|---|---|---|---|---|---|
| Sample Status | Sample ID | (raw) | (normalized) | Status | Promega |
| MSS | adenokEndocol_8792 | 0.01041 | 0 | neg | neg |
| MSS | colon_8953 | 0.02661 | 0 | neg | neg |
| MSS | colon_9215 | 0.03468 | 0.03761 | neg | neg |
| MSS | colon_9226 | 0.02467 | 0 | neg | neg |
| MSS | colon_9240 | 0.0033 | 0 | neg | neg |
| MSS | colon_9247 | 0.02576 | 0 | neg | neg |
| MSS | colon_9315 | 0.04469 | 0.15792 | neg | neg |
| MSS | colon_9316 | 0.0506 | 0.22897 | neg | neg |
| MSS | colon_9318 | 0.02599 | 0 | neg | neg |
| MSS | colon_9321 | 0.04278 | 0.13497 | neg | neg |
| MSS | colon_9328 | 0.05306 | 0.25857 | neg | neg |
| MSS | colon_9331 | 0.0278 | 0 | neg | neg |
| MSS | colon_9339 | 0.00479 | 0 | neg | neg |
| MSS | colon_9340 | 0.02461 | 0 | neg | neg |
| MSS | colon_9417 | 0.04382 | 0.14743 | neg | neg |
| MSS | colon_9467 | 0.04842 | 0.2027 | neg | neg |
| MSS | endometre_8510 | 0.06304 | 0.37852 | neg | neg |
| MSS | endometre_8729 | 0.01653 | 0 | neg | neg |
| MSS | endometre_9084 | 0.02216 | 0 | neg | neg |
| MSS | ovaire_8969 | 0.01892 | 0 | neg | neg |
| MSS | ovaire_9025 | 0.05002 | 0.22196 | neg | neg |
| MSI | HD_701 | 3.53191 | 1 | pos | pos |
| MSI | adenokEndometre_9304 | 0.35728 | 1 | pos | pos |
| MSI | endometre_7802 | 0.46849 | 1 | pos | pos |
| MSI | endometre_8283 | 0.29875 | 1 | pos | pos |
| MSI | endometre_8396 | 0.33278 | 1 | pos | pos |
| MSI | endometre_9179 | 0.51384 | 1 | pos | pos |
| MSI | endometre_9396 | 1.30956 | 1 | pos | pos |
| MSI | endometre_9462 | 0.78132 | 1 | pos | pos |
| MSI | endometre_9497 | 0.53486 | 1 | pos | pos |
| MSI | endometre_9500 | 0.85443 | 1 | pos | pos |
| MSI | uterus_9038 | 0.07979 | 0.57985 | pos | pos |

As reported in, the proposed NGS-based MSI Classifier also exhibits a lower limit of detection (LOD) compared to the Promega tests. For this experiment, 8 different dilutions of MSI-confirmed DNA sample and MSS-confirmed DNA samples have been tested, respectively comprising 1% (0.5 ng) to 90% (45 ng) of MSI-confirmed DNA in a sample of 50 ng mixed DNA content. On sample mixtures, the proposed NGS-based MSI Classifier provides the MSI status with down to 1% MSI tumor content mixed with 99% MSS content, while the Promega test only provides satisfactory results above a LOD of 20%.

TABLE 2
Comparison of limits of detection for the PROMEGA reference
test and the proposed NGS-based MSI-Score Classifier.

| | | MSI-Score | MSI-Score | MSI-Score | |
|---|---|---|---|---|---|
| Sample Status | Sample ID | (raw) | (normalized) | Status | Promega |
| 1% MSI + 99% MSS | LOD_01 | 0.1095 | 0.88154 | pos | neg |
| 2% MSI + 98% MSS | LOD_02 | 0.11381 | 0.9254 | pos | neg |
| 5% MSI + 95% MSS | LOD_05 | 0.1444 | 1 | pos | doubtful |
| 10% MSI + 90% MSS | LOD_10 | 0.18723 | 1 | pos | doubtful |
| 20% MSI + 80% MSS | LOD_20 | 0.34593 | 1 | pos | pos |
| 30% MSI + 70% MSS | LOD_30 | 0.48229 | 1 | pos | pos |
| 50% MSI + 50% MSS | LOD_50 | 0.64946 | 1 | pos | pos |
| 90% MSI + 10% MSS | LOD_90 | 1.27045 | 1 | pos | pos |

While the proposed methods and embodiments have been described so far for a simple positive/negative classification of the MSI status, the proposed NGS-based MSI Classifier may also report three different states of the MSI score:
positive (MSI-High) if the global score exceeds a first predefined cutoff value, for instance 0.1;
negative (MSI-Low) if the global score is below a second predefined cutoff value, for instance 0.05;
or in-between (MSI-Low) if the global score is between the first and the second cutoff values.

As will be apparent to those skilled in the art of bioinformatics, while the proposed methods and examples have been described primarily for microsatellite loci comprising homopolymer (mononucleotide) repeats, they may also be applied to microsatellite loci comprising heteropolymer or short tandem repeats, the repeat length distribution referring to the number of repeats of the heteropolymer sequences rather than the number of repeats of the homopolymer nucleotide.

As will be apparent to those skilled in the art of genomics, while the disclosed method does not require paired tumor-normal samples, the method is compatible with paired tumor-normal samples. While the envisioned reference repeat length distribution D_{MSS} is based on a reference population that differs from the tumor subject, D_{MSS} may also be inferred from a paired normal sample of the same subject. In a possible embodiment, the reference repeat length distribution D_{MSS} may be based on multiple non-tumor samples of the subject. In another possible embodiment, the reference repeat length distribution D_{MSS} may be based on a baseline sample in a serial biopsy dataset. It will be apparent that the model can easily be configured to detect decreases of the MSI score, for example following a treatment. The instant method may therefore support monitoring of the MSI status through longitudinal series.

As will be apparent to those skilled in the art of bioinformatics, a diversity of genomic data analyzer workflows may employ the proposed methods, possibly in combination with probabilistic sequencing and/or probabilistic variant calling methods. In a possible embodiment, a probabilistic classifier may be trained to calculate the global MSI score and/or to report the MSI status according to the local MSI scores across the N loci. In some possible embodiments, the probabilistic classifiers may take into account the read families based on unique molecular identifiers.

While the instant invention discloses the transformation of a quantitative MSI score into a qualitative MSI status, the quantitative MSI score obtained with the present method may be combined with other evidence when assessing the MSI status, or when predicting prognosis, treatment effects, and the like. In a possible embodiment, the MSI status may be computed through a combination of the MSI score computed using the method disclosed here and other evidence obtained from variant analyses of genes in the mismatch repair system, tumor mutational burden estimations, global or marker-specific hypo- or hyper-methylation, digital analysis of stained slides, and the like. In a possible embodiment, the MSI score computed using the method disclosed here may be integrated into multimodal machine learning models trained to predict the prognosis, clinical events, or treatment responses of subjects based on combinations of different genomic markers, in some case alongside imaging, clinical, pathological, proteomics, methylomics, and/or transcriptomics data.

What is claimed is:

1. A method for determining a microsatellite instability (MSI) score of a subject comprising:
a. obtaining nucleic acids from the subject;
b. preparing a plurality of nucleic acid fragments for high-throughput sequencing;
c. subjecting the prepared nucleic acid fragments to high-throughput sequencing;
d. cleaning and aligning, with a computer processor, the sequencing reads to a reference genome;
e. identifying, with a computer processor, the reads spanning at least one MSI locus;
f. computing, with a computer processor, a MSI score for each of the at least one MSI loci by performing the steps of:
i. recording the repeat length of each read spanning the MSI locus;
ii. establishing a repeat length distribution D_{MSI} based on the reads spanning the MSI locus;
iii. comparing the repeat length distribution D_{MSI} to a reference repeat length distribution D_{MSS}, wherein the comparing involves optimizing a model with at least three parameters that represent the biological events underlying MSI;
iv. computing a MSI score based on at least two of the optimized parameters;
g. aggregating the MSI scores across the at least one MSI loci to obtain a global MSI score; and
h. reporting the global MSI score to the user.

2. The method of claim 1, where the nucleic acid is genomic DNA or cell-free DNA extracted from the subject, wherein the subject is suffering from, or suspected to suffer from, cancer.

3. The method of claim 2, wherein the nucleic acid contains a mixture of cancerous cell DNA and normal cell DNA.

4. The method of claim 3, wherein comparing the repeat length distribution D_{MSI} to a reference repeat length distribution D_{MSS} involves estimating, with a computer processor, using a curve-fitting method, three independent scalars of a multi-parametric function.

5. The method of claim 4, wherein the three scalars represent respectively the shift in microsatellite length in cancerous cells compared to the reference, the variability of microsatellite lengths in cancerous cells compared to the reference, and the fraction of the sample that represents cancerous cells as opposed to normal cells.

6. The method of claim 1, wherein preparing the plurality of nucleic acid fragments for high-throughput sequencing involves ligating adaptors to end-repaired DNA fragments.

7. The method of claim 6, wherein endogenous molecular identifiers are used to assign reads to read families encompassing potential PCR duplicates.

8. The method of claim 6, wherein the adapters contain exogenous molecular identifiers.

9. The method of claim 8, wherein the exogenous molecular identifiers are used in combination with endogenous molecular identifiers to assign reads to read families encompassing potential PCR duplicates.

10. The method of claims 7 and 9, wherein establishing a repeat length distribution D_{MSI} based on the reads spanning the MSI locus involves computing the one or more most likely repeat lengths for each read family and computing D_{MSI} as the distribution of family-level repeat lengths.

11. The method of claim 1, wherein the global MSI score is transformed into a MSI status based on at least one threshold.

12. A computer system to implement the estimation of the MSI scores from claim 1.

13. A genomic data analyzer incorporating the computer system of claim 12, wherein the genomic data analyzer also performs the cleaning and aligning of the sequencing reads to a reference genome and the identifying of the reads spanning at least one MSI locus.
